SEQUENCE LISTING (5 sequences)

SEQ ID NO: 1 - protein, 14 aa, Clostridium tetani
YIKANSKFIGITEL

SEQ ID NO: 2 - protein, 16 aa, Artificial Sequence
  dTT831-844-Cys-beta-Ala
  MOD_RES (1)..(1): ACETYLATION
YIKANSKFIGITELCA

SEQ ID NO: 3 - protein, 9 aa, Artificial Sequence
  O-glycosylated peptide from S1 protein of SARS-CoV-2
VQPTESIVR

SEQ ID NO: 4 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 5 - protein, 14 aa, Artificial Sequence
  dTT830-843
QYIKANSKFIGITE

(19) United States
(12) Patent Application Publication
(10) Pub. No.: US 2024/0308991 A1
(43) Pub. Date: Sep. 19, 2024

(54) PROCESS FOR THE SYNTHESIS OF REACTIVE CARBOHYDRATES INCLUDING ANTIGEN PRECURSORS FOR CONJUGATION WITH A CARRIER MATERIAL

(71) Applicant: KORANEX CAPITAL, Montréal, Québec (CA)

(72) Inventors: Tze Chieh SHIAO, Montréal, Québec (CA); Serge MIGNANI, Chatenay-Malabry (FR); Serge MOFFETT, Montréal, Québec (CA); Ram VISHWAKARMA, Gurugram (IN)

(73) Assignee: KORANEX CAPITAL, Montréal, Québec (CA)

(21) Appl. No.: 18/272,470

(22) PCT Filed: Jan. 14, 2022
(86) PCT No.: PCT/CA2022/050054

§ 371 Date: Jul. 14, 2023

(30) Foreign Application Priority Data
Jan. 15, 2021 (IN) 202121001963

(51) Int. Cl. C07D 405/14

(52) U.S. Cl. CPC C07D 405/14

(57) ABSTRACT

Carbohydrate of Formula (A)

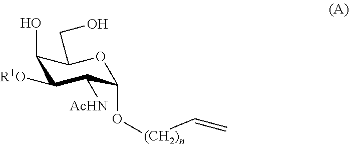

where R¹ is H and n an integer from 1 to 5, is synthesized by reacting, under heating, compound (A₀)

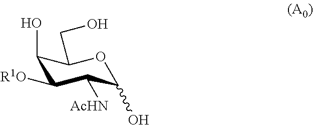

with HO—(CH₂)ₙ—CH═CH₂ in the presence of an acid capable of liberating a proton, and cooling the reaction mixture to obtain a cooled mixture comprising compound (A).

Carbohydrate of Formula (AA_{PG})

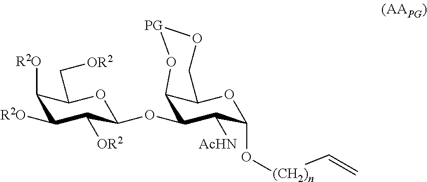

where R² is a monovalent protecting group, PG is a divalent protecting group and n an integer from 1 to 5, is synthesized by reacting, in the presence of a metal-containing zeolite, compound of Formula (A_{PG})

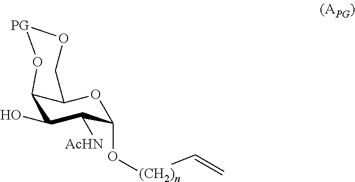

with compound of Formula (A₁)

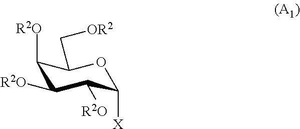

where X is —I, —Br, —Cl, —CN, —OTf (Triflate), —OMs (Mesylate), —OTs (Tosylate), methylsulfate, or trichloroacetimidate. Deprotection of compound (AA_{PG}) forms compound (AA)

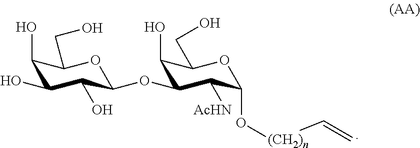

Carbohydrates (A) and (AA) can be coupled with a thiolated linker to form glycoconjugate precursors, which in turn are conjugatable with a carrier material such as a protein, peptide or polypeptide.

The present description relates to a process for the synthesis of reactive carbohydrates, such as antigen precursors, useful for conjugation with a carrier material (e.g., protein) and to a process for preparing glycoconjugates useful as immunogens and as therapeutic/diagnostic tools from the reactive carbohydrates.

The present description refers to a plurality of documents, the contents of which are herein incorporated by reference in their entirety.

### BACKGROUND

The ultimate objective of immunotherapy is to treat diseases like infections or cancers by modulating the innate and adaptive responses of the immune system to improve its ability to fight foreign substances such as bacteria, viruses, and cancer cells. Innate immunity is considered the first line of immune defense which is triggered in the early phases of exposure to pathogens. The cellular players include natural killer (NK) cells, dendritic cells (DCs), macrophages, monocytes, γδ T-cells and natural killer T (NKT)-cells. Unlike the innate immune system, adaptive immunity is slower to develop upon initial exposure to a foreign antigen but develops a highly specific response and creates immunological memory for a long-lasting protection. It involves the clonal expansion of T cells and B cells and their humoral and cellular mediators, cytokines and antibodies. The principal interfaces between the innate and adaptive immune responses are the professional antigen-presenting cells (pAPCs); macrophages, B cells and particularly dendritic cells (DCs). pAPCs are able to process and present antigens from endogenic and exogenic sources to T cells. They recognize microorganisms through pattern recognition receptors (PRRs) such as Toll-like receptors (TLRs). On recognition of microbial surface determinants or aberrant and unnatural antigens, the microorganisms or tumors and their related antigenic markers can be engulfed by the pAPC through an endocytic pathway where it is typically degraded into peptide fragments and the released antigen is bound onto intracellular MHC class I or class II molecules (pMHC). The pAPCs undergo maturation and activation leading to a redistribution of the pMHC complexes from intracellular compartments to the cell surface, secretion of cytokines and chemokines. In addition to pAPC, all nucleated cells types display only endogenous peptides on the cell membranes. In contrast to pAPC, these peptides originating from within the cell itself, including virus and intracellular pathogens, are displayed by MHC class I molecules coupled to b2-miccroglobulin. APC do not typically express MHC class II.

The peptides displayed on MHC class II molecules are typically recognized by the T cell antigen receptor (TCR) on CD4+ T helper cells which in turn undergo functional maturation into different subsets, such as Th1 or Th2 cells, upon co-stimulatory signals received from the pAPC. Th1 cells lead to a predominantly pro-inflammatory response with the secretion of IFN-γ and TNF-α, whereas Th2 cells secrete typical cytokines. Albeit Th1 cells are mainly associated with a cell-mediated response, both types of Th cells support the production of antibodies by B cells, which in turn influences antibody isotype and function. For example, IL-12 and TNF-α are associated with the differentiation of Th1 cells and production of type 1 IgG subclasses, whereas IL-6 and other Th2 cytokines contribute to the type 2 IgG subclass (IgG1) production.

The APC that display peptides on MHC class I molecules are recognized by the TCR of the CD8+ cytotoxic T cells. Several additional interactions between co-stimulator molecules expressed by the two cell types trigger the activation of the cytotoxic T cells into effector cells, while a strong and long lasting memory T cells is generated when dendritic cells interact with both the activated T-helper and the T-cytotoxic cells. Once activated, the T cell undergoes clonal selection and expansion with the help of the cytokine. This increases the number of cells specific for the dysfunctional target antigen can then travel throughout the body in search of the dysfunctional antigen-positive somatic cells. When docked onto the target cell, the activated cytotoxic T cell release payload of cytotoxins such as perforin and ganzymes. Through the action of perforin, granzymes penetrate the target cells and its proteases trigger the cell death. The cytotoxic T cell can also trigger the target cell death by the FAS signaling pathway.

It is thus desirable to be able to tailor vaccine-induced immunity to an appropriate response to deal with a pathogen or tumor antigen of interest.

Carbohydrates, as opposed to proteins and peptides, are T cell independent antigens not properly equipped to trigger the participation of Th cells and hence, cannot induce immune cell proliferation, antibody class switching, and affinity/specificity maturation. The major early advances initially encountered with carbohydrate-based vaccines have been supported by the discovery that, when properly conjugated to carrier proteins, serving as T cell dependent epitopes, bacterial capsular polysaccharides became capable of acquiring the requisite immunochemical ability to produce opsonophagocytic antibodies.

Traditionally, strategies for conjugating carbohydrate antigens to carrier proteins have relied on either reductive amination of aldehyde-derived sugars onto the ε-amino groups of the lysine residues, or simply amide coupling reactions. In both cases, partial and random carbohydrate antigen conjugation generally occurs. Furthermore, if all amide partners (amines from lysine or acid from glutamic/aspartic acids) are used for carbohydrate conjugation, far too many carbohydrate antigens become attached to the carrier proteins, thus resulting in masking potentially essential T cell peptide epitopes with the inherent diminution/elimination of immunogenicity. Thus, current strategies for preparing glycoconjugate vaccines are inadequate and face significant regulatory and/or commercial obstacles, since the preparations lack the necessary homogeneity in terms of their carbohydrate distribution and reproducibility (i.e., the attachment points of the sugars onto the proteins are randomly distributed and in various densities from batch to batch). Thus, glycoconjugate vaccines having greater carbohydrate antigen homogeneity, more precisely characterizable structures, and reproducibility from batch to batch would be highly desirable.

In addition, the synthesis of reactive carbohydrates useful as antigen precursors for conjugation with proteins, can present various challenges. Current synthetic pathways can be challenging in terms of stereoselectivity control, leading to the obtention of intermediate stereoisomers which can be difficult to separate and/or to the desired stereoisomers in low yields.

SARS-CoV-2, the causative agent of the COVID-19 pandemic that began in late 2019, represents an ongoing threat to global human health that has also crippled global economies. Initial vaccine development efforts have largely focused on protein antigens and epitopes present on the spike (S) glycoprotein, which mediates cell entry and membrane fusion of SARS-CoV-2 into host cells. However, global health experts have strongly recommended that scientists explore different strategies in parallel for developing therapeutic interventions against SARS-CoV-2 to mitigate against potential failures or complications that may arise for a single strategy, including new mutations in the virus that may evade current vaccine strategies directed at protein epitopes. Thus, there remains a need for developing vaccines and other therapeutic tools against SARS-CoV-2 in parallel to those focused on the protein antigens present on the S protein of SARS-CoV-2.

### SUMMARY

In a first aspect, described herein is a process for synthesizing a reactive carbohydrate of Formula (A)

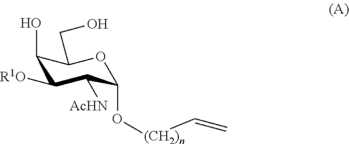

where R¹ is H and n is an integer from 1 to 5, comprising reacting, under heating, a compound of Formula (A₀)

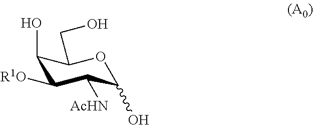

with HO—(CH₂)ₙ—CH═CH₂ in the presence of an acid capable of liberating a proton, resulting in a reaction mixture comprising the compound of Formula (A), and cooling the reaction mixture to obtain a cooled mixture comprising the compound of Formula (A).

In some embodiments, reacting the compound of Formula (A₀) comprises adding the compound of Formula (A₀) to a solution comprising HO—(CH₂)ₙ—CH═CH₂ and acetyl chloride at a mixing temperature from about 0° C. to about 25° C. resulting in an intermediate reaction mixture at low temperature comprising the compound of Formula (A₀), HO—(CH₂)ₙ—CH═CH₂ and HCl.

In a further aspect, described herein is a process for synthesizing a reactive carbohydrate of Formula (AA_{PG})

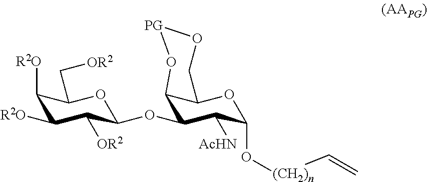

where R² is a monovalent protecting group, PG is a divalent protecting group and n is an integer from 1 to 5, comprising reacting, in the presence of a metal-containing zeolite, a compound of Formula (A_{PG})

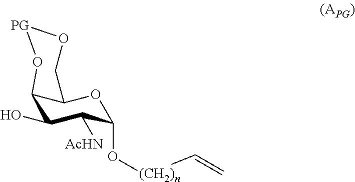

with a compound of Formula (A₁)

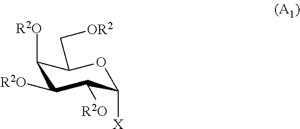

where X is —I, —Br, —Cl, —CN, —OTf (Triflate), —OMs (Mesylate), —OTs (Tosylate), methylsulfate, or trichloroacetimidate.

In some embodiments, the reaction between the compound of Formula (A_{PG}) and (A₁) is performed in the presence of a molecular sieve.

In some embodiments, the compound of Formula (A_{PG}) is formed by protecting a compound of Formula (A)

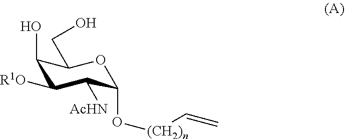

where R¹ is H, with the protecting group PG.
In some embodiments, the compound of Formula (A) is obtained by the process as defined herein.

In a further aspect, described herein is a process for synthesizing a reactive carbohydrate of Formula (AA)

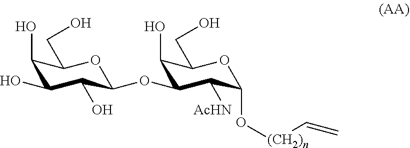

where n is an integer from 1 to 5, comprising deprotecting the compound of Formula (AA_{PG}) obtained by the process as defined herein.

In some embodiments, deprotecting comprises reacting the compound of Formula (AA_{PG}) with a basic solution to form an intermediate product, and then treating the intermediate product with an acidic solution.

In a further aspect, described herein is a process for synthesizing a reactive carbohydrate of Formula (AA)

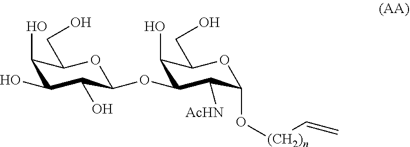

comprising a deprotection a compound of Formula (AA_{PG})

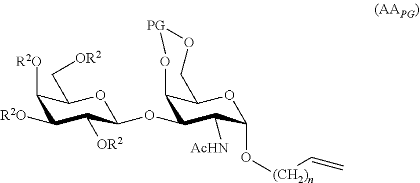

where n is an integer from 1 to 5, R² is a monovalent protecting group, and PG is a divalent protecting group, the deprotection comprising reacting the compound of Formula (AA_{PG}) with a basic solution to form an intermediate crude product, and then treating the intermediate crude product with an acidic solution.

In a further aspect, described herein is a process for synthesizing a reactive carbohydrate of Formula (B) or Formula (BB)

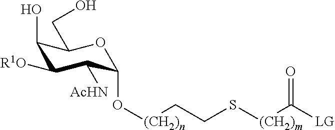

(B): R¹ is H
(BB): R¹ is

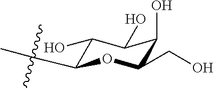

where n is an integer from 1 to 5, m is an integer from 1 to 5 and LG is a leaving group enabling an amido bond formation when the compound of Formula (B) or (BB) is reacted with an amino group, comprising reacting a compound of Formula (C)

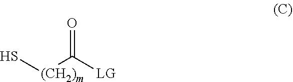

with a compound of Formula (A)

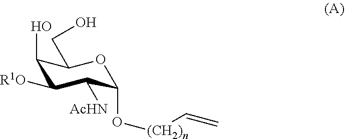

where R¹ is H, to form the compound of formula (B), or with a compound of Formula (AA)

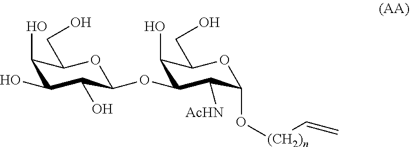

to form the compound of formula (BB).

In some embodiments, the compound of formula (A) is obtained by the process defined herein.

In some embodiments, the compound of formula (AA) is obtained by the process defined herein.

In some embodiments, reacting the compound of Formula (C) with the compound of Formula (A) or (AA) is performed in the presence of a photoinitiator.

In some embodiments, reacting the compound of Formula (C) with the compound of Formula (A) or (AA) is performed under ultraviolet light irradiation.

In some embodiments, reacting the compound of Formula (C) with the compound of Formula (A) or (AA) is performed under ultraviolet light irradiation in the presence of a photoinitiator.

In some embodiments, reacting the compound of Formula (C) with the compound of Formula (A) or (AA) is performed under visible light in the presence of a visible light absorbing transition metal photocatalyst.

In some embodiments, m in Formula (B), (BB) and/or (C) is an integer from 1 to 3. In some embodiments, m is 2.

In a further aspect, described herein is a process for preparing a glycoconjugate of Formula (I) or (II)

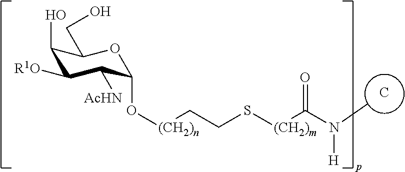

(I): R¹═H
(II): R¹═

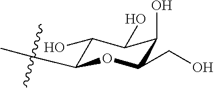

where n is an integer from 1 to 5, m is an integer from 1 to 5, p is an integer from 1 to 50 and

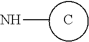

is a carrier material containing at least one amino group available for conjugation, comprising conjugating at least one free amino group of the carrier material with a compound of Formula (B) to form the glycoconjugate of Formula (I) or with a compound of Formula (BB) to form the glycoconjugate of Formula (I), wherein the compounds of Formula (B) and (BB) are obtained by the process defined herein.

In some embodiments, the carrier material comprises a protein, polypeptide or peptide. In some embodiments, in the process defined herein n is an integer from 1 to 3. In some embodiments, n is 1 or 2. In some embodiments, n is 1.

In a further aspect, described herein is a compound of Formula (AA_{PG})

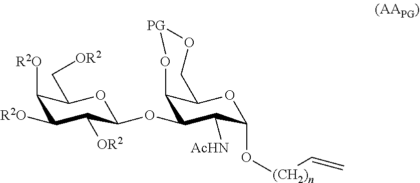

where n is an integer from 1 to 5, R² is Ac and PG is a divalent protecting group selected from PhCH, Me₂C, O═C, Me₂Si and 2,6-bis(trifluoromethyl)phenyl-B.

In some embodiments, PG in the compound (AA_{PG}) is PhCH.

In some embodiments, n in the compound (AA_{PG}) is an integer from 1 to 3. In some embodiments, n is 1 or 2. In some embodiments, n is 1.

In some embodiments, the compound (AA_{PG}) has the following formula:

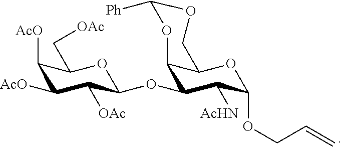

In a further aspect, described herein is a compound of formula:

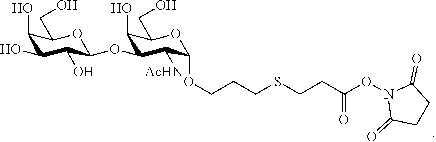

### General Definitions

Headings, and other identifiers, e.g., (a), (b), (i), (ii), etc., are presented merely for ease of reading the specification and claims. The use of headings or other identifiers in the specification or claims does not necessarily require the steps or elements be performed in alphabetical or numerical order or the order in which they are presented.

The use of the word “a” or “an” when used in conjunction with the term “comprising” in the claims and/or the specification may mean “one” but it is also consistent with the meaning of “one or more”, “at least one”, and “one or more than one”.

The term “about” is used to indicate that a value includes the standard deviation of error for the device or method being employed in order to determine the value. In general, the terminology “about” is meant to designate a possible variation of up to 10%. Therefore, a variation of 1, 2, 3, 4, 5, 6, 7, 8, 9 and 10% of a value is included in the term “about”. Unless indicated otherwise, use of the term “about” before a range applies to both ends of the range.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The expression “leaving group” or “LG” as used herein refers to a leaving group that provides improved reaction efficiency and/or specificity (i.e., improved conjugation to a free amine group of a polypeptide or protein) as compared to the corresponding functional group prior to replacement with the leaving group. In some embodiments, the leaving groups may be an O-fluorophenyl group such as OPhF₅ or OPhF₄(para SO₃Na), or an O—(N-succinimidyl) group.

As used herein, the term “carrier material” refers to a material that is capable to be conjugated to a carbohydrate antigen (e.g., an antigenic monosaccharide, di-saccharide, oligo-saccharide, or polysaccharide, e.g. a natural or synthetic antigen). In some embodiments, the carrier material can be conjugatable to the carbohydrate antigen through a thiol-linker that is terminated with a group capable of forming an amido bound with at least one amino present on the carrier material. In some embodiments, the carrier material can be a carrier protein. In other embodiments, the carrier material may comprise a sensor chip, microassay or beads, to name a few examples, which bear amino groups.

As used herein, the term “protein” (e.g., in the expression “carrier protein”) means any peptide-linked chain of amino acids, which may or may not comprise any type of modification (e.g., chemical or post-translational modifications such as acetylation, phosphorylation, glycosylation, sulfatation, sumoylation, prenylation, ubiquitination, etc.), so long as the modifications do not destroy the immunogenicity of the glycoconjugate immunogens and glycoconjugate vaccines described herein. For further clarity, the terms “protein” and “carrier protein” as used herein encompass both peptides and polypeptides, even though both embodiments may be recited together such as in the expression “carrier protein(s) or peptide(s)”.

As used herein, the term “glycoconjugate” refers to a carbohydrate antigen (e.g., an antigenic monosaccharide, di-saccharide, oligo-saccharide, or polysaccharide, e.g. a natural or synthetic antigen) coupled to a carrier material. When the carrier material is a carrier protein or peptide, the coupling can enhance the immunogenicity of carbohydrate antigen in a subject of interest. The expressions “carbohydrate antigen” and “sugar antigen” carry the same meaning as used herein. The term “immunogen” refers to an agent that is capable of being specifically bound by components of the immune system (e.g., by an antibody and/or lymphocytes), and generating a humoral and/or cell-mediated immune response in a subject of interest. As used herein, the term “immunogen” in an expression such as “glycoconjugate immunogen” refers to the ability (i.e., physical characteristic or property) of the glycoconjugate without limiting the glycoconjugate itself to a particular use (e.g., as an immunogen for generating an immune response in a subject). For example, in some embodiments, a glycoconjugate immunogen described herein may be employed in diagnostic assays or methods (e.g., in vitro methods) to detect the presence or absence of an antibody that binds to the glycoconjugate immunogen in a biological sample (e.g., from a subject). In some embodiments, the glycoconjugate immunogens described herein may be used for screening, identifying, or evaluating antibodies that bind specifically to the glycoconjugate immunogen (e.g., monoclonal antibodies that are diagnostically or therapeutically applicable).

As used herein, the term “synthetic” refers to a compound that is not a product of nature, which is produced by human intervention.

As used herein, the term “conjugatable” refers to the ability or capability of at least two molecules (e.g., a carbohydrate antigen and a thio-linker; or a carbohydrate antigen and a carrier material such as a protein or peptide) to be covalently bonded to one another via a chemical reaction, regardless of whether the molecules are actually covalently bonded to one another. In contrast, the term “conjugated” refers to at least two molecules (e.g., a carbohydrate antigen and a thio-linker, or a carbohydrate antigen and a carrier material such as a protein or peptide) which are covalently bonded to one another.

As used herein, the term “administration” may comprise administration routes such as parenteral (e.g., subcutaneously, intradermally, intramuscularly, or intravenously), oral, transdermal, intranasal, etc., so long as the route of administration results in the generation of an immune response in the subject.

As used herein, the term “subject” generally refers to a living being (e.g., animal or human) that is able to mount an immune response to a glycoconjugate as described herein, preferably leading to the production of antibodies and/or lymphocytes that specifically bind to the glycoconjugate and/or cells presenting the glycoconjugate. In some embodiments, a subject described herein may be a patient to be treated therapeutically (e.g., via vaccination with a glycoconjugate immunogen described herein) or may be employed as a means for generating tools (e.g., antibodies) for research, diagnostic, and/or therapeutic purposes.

Signification of various abbreviations used in the present disclosure are provided in Table 1 below.

TABLE 1

| Abbreviation | Signification |
|---|---|
| ACN | Acetonitrile |
| ACVA | 4,4′-azobis(4-cyanovaleric acid) |
| BSA | Bovine serum albumin |
| calcd | calculated |
| CRM197 | Cross-reacting material 197 |
| COSY | COrrelated SpectroscopY |
| DCM | Dichloromethane |
| DMF | Dimethylformamide |
| ELISA | Enzyme-linked immunosorbent assay |
| equiv. | equivalent |
| ESI⁺-HRMS | Electrospray Ionization High Resolution Mass spectrometry |
| HSQC | Heteronuclear Single Quantum Coherence |
| HPLC | High-Performance Liquid Chromatography |
| GalNAc | N-acetylgalactosamine |
| LC-MS-TOF | Liquid Chromatography-Mass Spectrometry-Time Of Flight |
| LED | Light-emitting diode |
| MALDI- | Matrix Assisted Laser Desorption Ionization-Time Of Flight |
| TOF | |
| mol. sieve | molecular sieve |
| NHS | N-Hydroxysuccinimide |
| NMR | Nuclear Magnetic Resonance |
| PBS | Phosphate-buffered saline |
| PNA | Peanut Agglutinin |
| Rf | Retention factor |
| SDS-PAGE | Sodium dodecyl sulphate-polyacrylamide gel |
| | electrophoresis |
| TMSOTf | Trimethylsilyl trifluoromethanesulfonate |

Other objects, advantages and features of the present description will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1 shows the results of an ELISA that shows the reactivity of the TF-specific lectin Peanut Agglutinin (PNA) and anti-TF monoclonal antibody JAA-F11 to 0.01 μg of unconjugated CRM197 and CRM197 conjugated to TF (CRM-TF: glyconjugate (II)) at 3 different ratios (0.2, 0.6, 3) of NHS-TF (compound (11b)) to the protein's total number of lysine.

FIG. 2 shows a Coomassie stained SDS-PAGE gel of 1 μg of unconjugated CRM197 and CRM197 conjugated to TF (CRM-TF: glyconjugate (II)) at 3 different ratios of NHS-TF (compound (11b)) to the protein's total number of lysine.

FIG. 3 shows a Western blot of 1 pg of unconjugated CRM197 and CRM197 conjugated to TF (CRM-TF: glyconjugate (II)) at 3 different ratios of NHS-TF (compound (11b)) to the protein's total number of lysine detected using the TF-specific lectin Peanut Agglutinin (PNA) and anti-TF monoclonal antibody JAA-F11.

FIG. 4 shows the overlay of MALDI-TOF spectra of unconjugated CRM197 and CRM197 conjugated to TF (CRM-TF: glyconjugate (II)) at 3 different ratios of NHS-TF (compound (11b)) to the protein's total number of lysine.

### SEQUENCE LISTING

This application contains a Sequence Listing in computer readable form created Jan. 11, 2022 having a size of about 15 Kb. The computer readable form is incorporated herein by reference.

### DETAILED DESCRIPTION

The present description relates to a process for synthesizing reactive carbohydrates, including antigen precursors, as well as glycoconjugates suitable for use such as immunogens, in vaccines, in diagnostics, or for generating analytic or therapeutic tools (e.g., generating novel anti-glycoconjugate antibodies).

Conjugating carbohydrate antigens ending in terminal acid functionalities to amine groups of carrier proteins is traditionally done through random activation with succinimide or carbodiimide reagents. One of the major disadvantages of such carbohydrate antigen-carrier protein conjugation methods is the uncontrolled/undesired self-crosslinking that occurs within the carrier protein itself, wherein the side chains of the carrier protein's own aspartic/glutamic acid residues become coupled to the ε-amine groups of the carrier protein's own lysine residues. This approach leads to perturbation or destruction of the native structure of the carrier protein, often resulting in substantial loss of key peptide sequences that would otherwise be highly immunogenic, as well as potential undesirable cross-linking of the carrier protein. In addition, carbohydrate antigen-carrier protein conjugation methods described in the art often employ linkers such as squaric acids and the like, that may trigger immune responses against the linkers themselves rather than to only the carbohydrate antigens to which they are coupled. Furthermore, carbohydrate antigen-carrier protein conjugation methods described in the art do not allow for adequate control over the extent to which the carrier proteins are glycosylated, often resulting in heterogenous glycoconjugate species, which is a significant barrier to production for human therapeutics. In addition, the synthesis of reactive carbohydrate intermediates useful as antigen precursors for conjugation with proteins, can present various challenges. Current synthetic pathways can be challenging in terms of stereoselectivity control, leading to the obtention of intermediate stereoisomers which can be difficult to separate and/or to the desired stereoisomers in low yields.

In contrast, the present description provides for processes allowing the synthesis of intermediate reactive carbohydrates with better stereoselectivity control and resulting in synthetic antigens suitable for conjugation with a carrier material such as a carrier protein, in fewer steps than currently known processes. The present description also provides for a process for synthesising glycoconjugates using simple and limited synthetic steps while still allowing adequate control over the extent to which the carrier material is glycosylated.

In a first aspect, the present description relates to an improved process for producing reactive carbohydrates which are conjugatable with a carrier material (e.g., carrier protein or peptide) having one or more free amine groups to result in glycoconjugates. The reactive carbohydrates may be purified and subsequently employed in the coupling reaction with the carrier material having one or more free amine groups. The coupling reaction conjugates one or more of the purified reactive carbohydrates to the carrier material at the one or more free amine groups via an amide bond, thereby producing the glycoconjugate.

In a further aspect, described herein is a process for producing a glycoconjugate from the synthetic reactive carbohydrates prepared according to the process described herein.

In another aspect, described herein are processes for producing intermediate reactive carbohydrates which can be reacted to obtain the conjugatable reactive carbohydrates.

The following Scheme 1 presents synthetic steps that may be involved in these various processes.

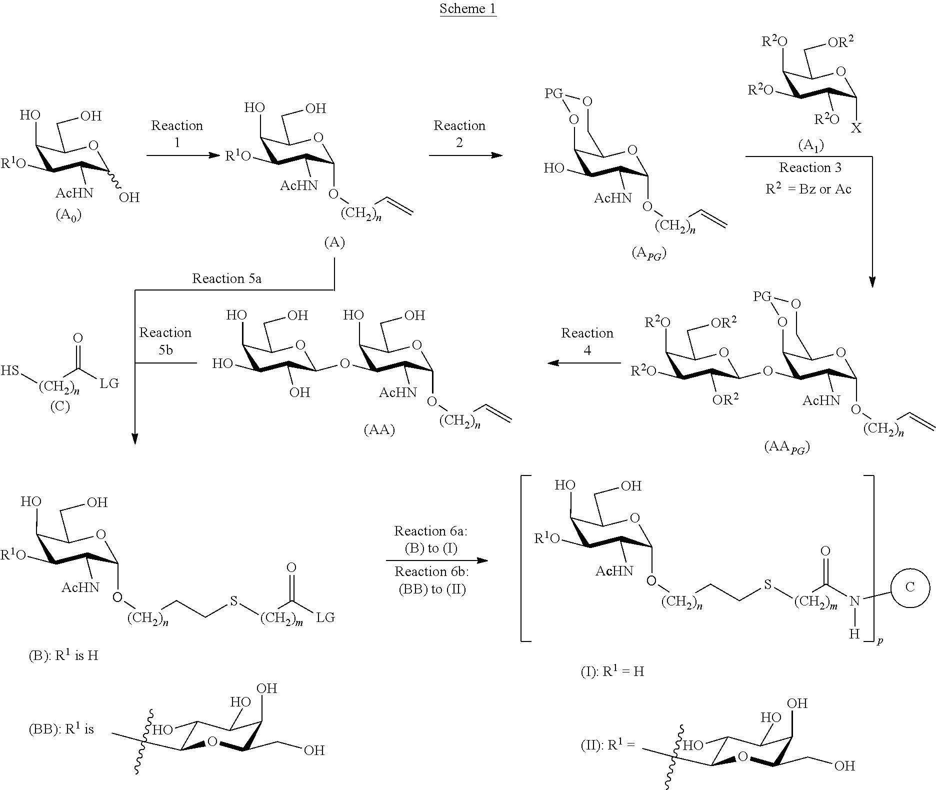

### Process Reaction 1

In some embodiments, there is first provided the synthesis of the reactive carbohydrate of Formula (A)

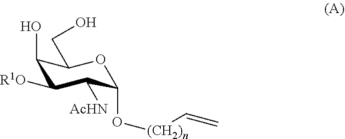

where R¹ is H and n is an integer from 1 to 5, e.g., from 1 to 3, preferably 1 or 2, most preferably 1. The process can comprise a reaction between a compound of Formula (A₀)

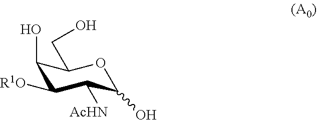

with an alcohol of formula HO—(CH₂)ₙ—CH═CH₂ in the presence of an acid. The reaction is performed under heating, to result in a reaction mixture comprising the compound of Formula (A), that can then be cooled. The synthesis of the compound of Formula (A) thus involves an etherification on the C₁ carbon of the carbohydrate (A₀) in acidic conditions with the alcohol HO—(CH₂)ₙ—CH═CH₂. Several acids can be used for performing the reaction as long as the acid is capable of liberating a proton H⁺ in the reaction mixture for catalyzing the reaction which can lead to the formation of the compound of Formula (A). In some embodiments, the acid can be a strong acid or a Lewis acid. In some embodiments, the Lewis acid can be different than BF₃·Et₂O.

In some embodiments, the acid used in the reaction to form the compound of Formula (A) from compound (A₀) and the alcohol HO—(CH₂)ₙ—CH═CH₂ can be acetyl chloride, acetic acid, an acidic cation exchange resin, camphorsulfonic acid, p-toluenesulfonic acid monohydrate, HCl or any mixture thereof.

In some embodiments, the compound of Formula (A₀) is reacted with the alcohol HO—(CH₂)ₙ—CH═CH₂ in the presence of acetyl chloride as the acid. A solution comprising HO—(CH₂)ₙ—CH═CH₂ and acetyl chloride can be first prepared at low temperature, such as a temperature from about 0° C. to about 25° C. In some implementations, the solution comprising HO—(CH₂)ₙ—CH═CH₂ and acetyl chloride can be prepared at a temperature that is about 0° C. In other embodiments, the temperature at which the solution of HO—(CH₂)ₙ—CH═CH₂ and acetyl chloride is prepared can be from about 0° C. to about 10° C., or about 0° C. to about 20° C., or about 10° C. to about 20° C., or about 10° C. to about 25° C., or any temperature comprised in these ranges. When preparing the solution of HO—(CH₂)ₙ—CH═CH₂ and acetyl chloride, an excess of the alcohol can be used. Hence, the alcohol can be used as reactant while also serving as solvent. When mixing the alcohol and the acetyl chloride, some HCl is formed together with some AcO—(CH₂)ₙ—CH═CH₂. The solution can preferably be prepared at low temperature to maintain some HCl in solution in the alcohol. In some embodiments, the solution of HO—(CH₂)ₙ—CH═CH₂ and acetyl chloride can be prepared under inert atmosphere, such as under argon or N₂. After completing the preparation of the solution of HO—(CH₂)ₙ—CH═CH₂ and acetyl chloride, the compound of Formula (A₀) is added to the solution, which can still be at low temperature, e.g., between about 0° C. to about 25° C., to form an intermediate reaction mixture at low temperature comprising at least the compound of Formula (A₀), HO—(CH₂)ₙ—CH═CH₂, HCl and some AcO—(CH₂)ₙ—CH═CH₂. Then, this intermediate reaction mixture is heated to promote the formation of the compound of Formula (A). Upon heating, the reaction between the compound of Formula (A₀) and HO—(CH₂)ₙ—CH═CH₂ can occur quickly in the presence of the proton H⁺ thereby forming the ether of Formula (A). In addition to activate the oxygen atom in the C₁ position of the compound (A₀), the protons can also activate the acetamido group on the carbon in position 2 of the sugar, increasing the anomeric effect in favor of the alpha conformation. The reaction in the presence of an acid such as acetyl chloride can thus promote the formation of the compound of Formula (A), having an alpha conformation, using a single reaction, as opposed to prior synthetic processes requiring protection-deprotection steps to arrive at the same compound. In some embodiments, the heating of the intermediate mixture can be performed at a temperature from about 40° C. to about 80° C. In some embodiments, the reaction temperature can be from about 60° C. to about 80° C., for instance it can be about 70° C. Any temperature comprised in these temperature ranges can be used as the heating temperature. The reaction time can be comprised between about 30 minutes to 5 hours. In some embodiments, the reaction time can be from about 30 min to about 1 hour. Using acetyl chloride as the acid, short reaction times can be sufficient, such as between about 30 min to about 45 min. Even just about 30 minutes can be enough. In some embodiments, the advancement of the reaction can be monitored, and the reaction time adjusted in consequence. In further embodiments, the intermediate mixture comprising at least the compound of Formula (A₀), HO—(CH₂)ₙ—CH═CH₂ and HCl at low temperature is contacted with the heat source at a temperature high enough to rapidly and immediately heat the mixture. In other words, in some embodiments, the intermediate mixture at low temperature can be put into contact with the heat source straight away, as opposed to a gradual heating from the low temperature to the heating temperature. At a laboratory scale, this could be performed by placing the round-bottom flask comprising the intermediate mixture at low temperature into an oil bath already at the heating temperature, as opposed to placing the round-bottom flask into an oil bath at low temperature and then heating the bath oil to increase the temperature and reach the desired heating temperature. At an industrial scale, any conventional heating means allowing for “contacting” the intermediate mixture with the heat source could be used. For instance, these could include heat exchangers. In some embodiments, the heat source can be at a temperature of from about 40° C. to about 80° C.

In further embodiments, the acid source capable of liberating protons for catalyzing the reaction for obtaining the compound of Formula (A), can comprise and acid selected from acetic acid, an acidic cation exchange resin, camphorsulfonic acid, p-toluenesulfonic acid monohydrate, HCl or any mixture thereof. In some embodiments, the reaction with these other types of acids can be performed by preparing a solution comprising the compound of Formula (A₀), HO—(CH₂)ₙ—CH═CH₂ and the acid, and then heating the solution. The solution can also comprise an organic solvent. In some embodiments, the solvent can be selected from dichoromethane, chloroform, 1,3-dioxolane, diethoxymethane, dimethoxymethane, 2,5,7,10-tetraoxaundecane, dipropoxymethane, just to name a few, and any combination thereof. In some embodiments, the reaction mixture comprising the compound of Formula (A₀), HO—(CH₂)ₙ—CH═CH₂ and the acid, can be heated at a temperature of from about 30° C. to about 70° C. Any temperatures within this range can be used. The reaction time can vary and monitoring the advancement of the reaction can allow to adjust the reaction time. In some embodiments, the reaction can be performed for a reaction time of about 0.5 hour to about 15 hours, or from about 0.5 hour to about 10 hours, or from about 0.5 hour to about 5 hours, or from about 0.5 hour to about 2 hours.

In some embodiments, the reaction temperature and reaction time for preparing the compound of Formula (A) from the compound (A₀), can further be adjusted by monitoring the formation of the equivalent beta conformer of the compound of Formula (A). Even if the above-mentioned reaction conditions (i.e., choice of the acid, temperatures, reaction times and/or intermediate mixture preparation) should promote the formation of the alpha conformer compound (A), some beta conformer can be formed during the reaction. By analyzing the reaction mixture during heating, one can assess the formation of the beta conformer and immediately stop heating and cool the mixture if some beta conformer is observed.

Once the heating step is completed and the reaction mixture is cooled, the cooled mixture can be neutralized by any conventional method (e.g., adding a base and checking the pH until reaching a pH of about 7). The compound of Formula (A) can then be isolated and purified by any conventional methods. For instance, the isolated crude product can be lyophilized, reprecipitated or purified by chromatography. In some embodiments, the purification step can allow separating the compound of Formula (A) from the equivalent beta conformer is some was formed during the reaction.

### Process Reaction 2

In some embodiments, there is also provided the synthesis of the compound of formula (A_{PG})

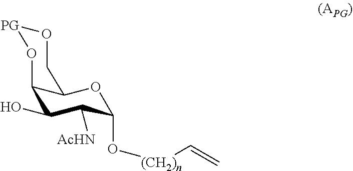

where PG is a protecting group and n is an integer from 1 to 5. The reaction comprises protecting a compound of formula (A)

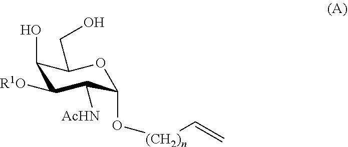

where R¹ is H, with the divalent protecting group PG.

In some embodiments, n is an integer from 1 to 3, preferably 1 or 2, most preferably 1.

In some embodiments, the compound of Formula (A) can be prepared by the Process Reaction 1 described above. In further embodiments, the protection of the compound of Formula (A) can be performed by reaction with a compound selected from the group consisting of PhCH(OMe)₂, triphosgene, Me₂SiCl₂, Me₂C(OMe)₂, 2-methoxypropene and 2,6-bis(trifluoromethyl)phenylboronic ester. Hence, in some embodiments, the reaction allows preparing a protected compound of Formula (A_{PG}) where PG is selected from the group consisting of PhCH, Me₂C, O═C, Me₂Si and 2,6-bis(trifluoromethyl)phenyl-B. In a preferred embodiment, the protecting group is PhCH.

In some embodiments, the protection reaction can be performed in an organic solvent selected from the group consisting of DMF, Dimethyl acetamide (DMAC), DMSO, THF, Dioxane, 1-3 Dioxolane, acetonitrile, 2,5-dimethyl tetrahydrofuran (DMTHF), Gamma-vaterolactone (GVL), Dihydrolevoglucoserone (Cyrene), methyl levulinate (ML), Ethyl levulinate (EL), Ethyl levulinate propyleneglycol ketal (ELPK), Dimethyl glutarate (DMG), Dimethylpropylene urea (DMPU), Poly(propyleneglycol) (PPG), Glycofurol (THFP), 1-Ethyl-3-methylimidazolium acetate ([emim][OAc]) and any combination thereof.

In some embodiments, the compound of Formula (A_{PG}) can be prepared by reacting a compound of Formula (A) with PhCH(OMe)₂ in DMF in the presence of a catalytic amount of p-toluenesulfonic acid monohydrate.

In some embodiments, the reaction can be performed at room temperature and the formation of the compound of Formula (A_{PG}) can be monitored and the reaction stopped when the reaction is substantially completed. In some embodiments, the reaction time can be about 5 hours. The compound can then be isolated by conventional methods.

### Process Reaction 3

In some embodiments, there is also provided the synthesis of the reactive carbohydrate compound of Formula (AA_{PG})

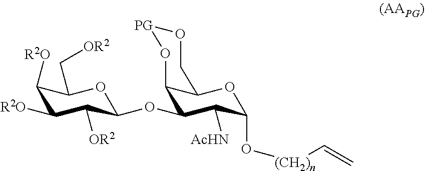

where R² is a monovalent protecting group, PG is a divalent protecting group and n is an integer from 1 to 5.

In some embodiments, the synthesis comprises a glycosylation, in the presence of a metal-containing zeolite, between a compound of Formula (A_{PG})

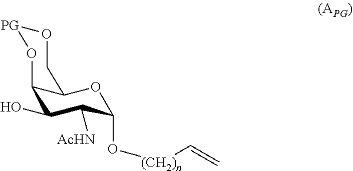

with a compound of formula (A₁)

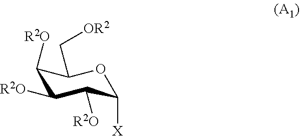

where X is —I, —Br, —Cl, —CN, —OTf (Triflate), —OMs (Mesylate), —OTs (Tosylate), methylsulfate, or trichloroacetimidate.

In some embodiments, the compound of Formula (A_{PG}) can be prepared by the Process Reaction 2 described above.

In some embodiments, the reaction to prepare the compound of Formula (AA_{PG}) can be performed in an organic solvent. For instance, the organic solvent can be selected from the group consisting of toluene, dichorometane-toluene mixture, DMSO-toluene mixture, and N-methylpyrrolidone-toluene mixture. In some embodiments, the solvent can be toluene. The reaction can be preferably performed under inert atmosphere (e.g. under argon or N₂).

In some embodiments, the monovalent protecting group R² can be selected from the group consisting of Ac, Bz, allyl, benzoate, methoxymethyl (MOM), tetrahydropropyranyl (THP), t-butyl, pivalate, t-butyldimethylsilyl (TBDMS) and t-butyldiphenylsilyl (TBDPS). Any other possible hydroxyl protecting groups can also be used. In preferred embodiments, R² can be Ac or Bz.

As previously mentioned, in some embodiments, the divalent protecting group PG, can be selected from PhCH, Me₂C, O═C, Me₂Si and 2,6-bis(trifluoromethyl)phenyl-B, preferably PhCH. The leaving group X in the compound of Formula (A₁) can preferably be a halogen selected from I, Br, or Cl, most preferably Br.

In some embodiments, n is an integer from 1 to 3, preferably 1 or 2, most preferably 1. In some embodiments, the metal-containing zeolite can be a zeolite having at least one metal which is part of the zeolite framework itself or having at least one metal present in the zeolite pores and/or at the zeolite surface. In some embodiments, the zeolite can thus be a metal-impregnated zeolite. In further embodiments, the metal in the metal-containing zeolite can comprise Ag, Al, Cd, Co, Cu, Fe, Ga, In, Mo, Pd, Pt, Sn, Sb, V, Zr or any mixture thereof. In some embodiments, the metal-containing zeolite can be a zeolite selected from the group consisting of Sn-Beta, Zr-Beta, Al-Beta(OH), Al-Beta(F), Pt@MCM-22, K-PtSn/MFI, 0.3Pt/0.5Sn—Si-Beta, Pt/Sn 2.0-Beta, 0.5CoSi-Beta, V-Beta, H—[Fe]ZSM-5, Fe-BEA, Ga-Beta, Ga-Beta-200, Mo/HZSM-5, or any mixture thereof, to name a few examples. However, any type of metal-containing zeolite capable of catalyzing the ether bond reaction between the compound of Formula (A_{PG}) and the compound of formula (A₁) can be used to prepare the compound of Formula (AA_{PG}). In preferred embodiments, the metal-containing zeolite can comprise a silver-containing zeolite such as a silver-exchanged zeolite for instance. Such zeolites are generally commercially available.

In some embodiments, the glycosylation reaction performed in the presence of the metal-containing zeolite can be done at room temperature or under heating. In some embodiments, the glycosylation temperature can be from about 20° C. to about 80° C. In other embodiments, the reaction is performed at a temperature from about 20° C. to about 70° C., from about 20° C. to about 60° C., from about 20° C. to about 50° C., from about 20° C. to about 40° C., or from about 20° C. to about 30° C. The glycosylation reaction can thus be performed at any temperature comprised within these ranges. In further embodiments, the glycosylation can be performed for a period of about 5 hours to about 50 hours. In other embodiments, the glycosylation time can last from about 5 hours to about 30 hours, or from about 5 hours to about 25 hours, from about 5 hours to about 20 hours, or from about 10 hours to about 30 hours, or from about 10 hours to about 25 hours, or from about 10 hours to about 20 hours, or from about 15 hours to about 30 hours, or from about 15 hours to about 25 hours, or from about 15 hours to about 20 hours, or from about 20 hours to about 25 hours, or from about 20 hours to about 30 hours. In some embodiments, the glycosylation reaction can be performed at about 60 to about 80° C. for about 15 hours to about 20 hours. In other embodiments, the glycosylation can be performed at about 20° C. to about 25° C. for about 20 hours to about 30 hours.

The use of a metal-containing zeolite as catalyst for the reaction between the two carbohydrates of Formulae (A_{PG}) and (A₁) surprisingly allowed to essentially obtain a glycosylated product (AA_{PG}) with the beta conformation between the two sugars. This is an important advantage compared to conventional glycosylation reactions (e.g., using trichloroacetimidate and BF₃·Et₂O or TMSOTf) which generate mixtures of the alpha and beta conformers. In the presently described process, the purification of the resulting product (AA_{PG}) can be easily performed by filtration to remove the zeolite at the end of the reaction. Furthermore, the glycosylation in presence of the zeolite can be easier to control than conventional reactions. For instance, the glycosylation in the presence of trichloroacetimidate and BF₃·Et₂O or TMSOTf may require maintaining the reaction temperature very low, such as at about −30° C., which in turn can slow down the reaction. Furthermore, the use of a metal-containing zeolite is preferable to the use of other catalysts such as Hg(CN)₂, also known to catalyze the glycosylation, but which includes a poisonous heavy metal and is therefore toxic. Overall, the use of a metal-containing zeolite allows performing the glycosylation more smoothly with high control of the stereoselectivity.

In some embodiments, the preparation of the compound of Formula (AA_{PG}), i.e. the coupling between the compounds of Formula (A_{PG}) and (A₁) can further be performed in the presence of a molecular sieve. Examples of molecular sieves that can be used include molecular sieves of type 3 Å, type 4 Å, type 5 Å, type 13X. Any mixture thereof can also be used. Performing the reaction in the presence of a molecular sieve can allow to trap moisture/traces of water, which can in turn promote the reaction.

As explained above, the final crude product (AA_{PG}) can be easily isolated by filtering the reaction mixture and then removing the solvent. In some embodiments, the crude product can then be purified by any conventional methods.

### Process Reaction 4

In some embodiments, there is also provided the synthesis of the reactive carbohydrate compound of Formula (AA)

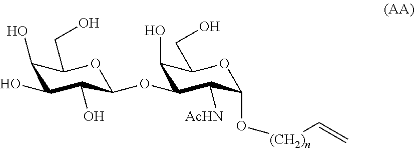

where n is an integer from 1 to 5.

The reaction can comprise deprotecting a compound of Formula (AA_{PG}) as described herein.

In some embodiments, the compound of Formula (AA_{PG}) can be obtained by the process as described herein.

In some embodiments, n is an integer from 1 to 3, preferably 1 or 2, most preferably 1.

In some embodiments, the deprotection of the compound of Formula (AA_{PG}) can comprise reacting the compound of Formula (AA_{PG}) with a basic solution to form an intermediate product (AAi), and then treating the intermediate product (AAi) with an acidic solution.

In some embodiments, the basic solution required to obtain the intermediate product (AAi) can comprise a base that is selected from the group consisting of a methoxide, ethoxide and 2-methylpropan-2-olate. In a preferred embodiment, the base can be sodium methoxide. In some embodiments, the basic solution can comprise a solvent selected from the group consisting of methanol, ethanol and isopropanol. In some embodiments, the solvent can preferably be methanol. In some embodiments, the reaction of (AA_{PG}) with the basic solution to form the intermediate product (AAi) can be performed at a temperature of from about 0° C. to about 50° C., preferably at room temperature. The reaction with the basic solution to form the intermediate product (AAi) can be performed for a period of about 0.5 hour to about 5 hours, preferably about 1 hour to about 2 hours.

In some embodiment, the intermediate product (AAi) in crude form can be directly treated with the acidic solution, without requiring any purification step. This is advantageous as it can reduce the number of steps of the protection process. In some embodiments, both the basic treatment and the subsequent acidic treatment can thus be performed in the same reaction vessel. In some embodiments, once the basic treatment is completed, the solution can be neutralized, and the liquid can be evaporated to afford the intermediate product (AAi) in crude form. Then, the intermediate product (AAi) in crude form can be directly treated with the acidic solution, without requiring any purification step as mentioned above.

In some embodiments, the subsequent acidic treatment can be performed by reacting the intermediate product (AAi) with an acidic solution comprising an acetic acid-water mixture or an ethanolic acid-water mixture. In a preferred embodiment, the acidic solution can be an aqueous acetic acid solution. In further embodiments, the intermediate product (AAi) can be treated with the acidic solution at a temperature of from about 50° C. to about 70° C., preferably at about 60° C. The treatment with the acidic solution can be performed for a period of about 1 hour to about 5 hours, preferably about 1 hour to about 2 hours, to result in a solution comprising the compound of Formula (AA).

In some embodiments, the compound of Formula (AA) can then be isolated by conventional methods including for instance extraction, precipitation, crystallisation, lyophilisation, chromatography on silica gel under normal phase or reverse phase on C-18, simulated moving bed (SMB) chromatography, and/or purification by membrane technology such as membrane nanofiltration.

### Process Reaction 5a and 5b

In some embodiments, there is also provided a process for synthesizing a reactive carbohydrate of Formula (B) or Formula (BB)

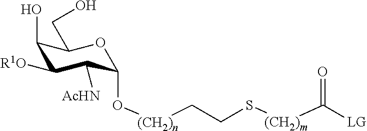

(B) R¹ is HH
(BB): R¹ is

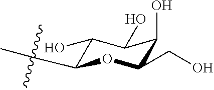

where n is an integer from 1 to 5, m is an integer from 1 to 5 and LG is a leaving group enabling an amido bond formation when the compound of Formula (B) or (BB) is reacted with an amino group. The compounds of Formula (B) or (BB) are also referred to as carbohydrate antigen precursors in the present description. The compounds of Formula (B) or (BB) comprise a thiol-linker to which a carbohydrate antigen is linked.

In some embodiments, the process for obtaining the compound of Formula (B) can comprise reacting a compound of Formula (C)

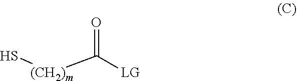

where m and LG are as defined above,
   with a compound of Formula (A)

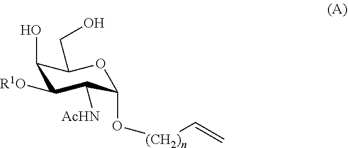

where R¹ is H and n is as defined above.

In some embodiments, the compound of formula (A) can be obtained by the process as defined hereinabove.

In some embodiments, the process for obtaining the compound of Formula (BB) can comprise reacting the compound of Formula (C) with a compound of Formula (AA)

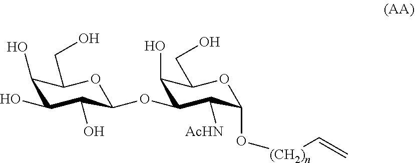

where n is as defined above.

In some embodiments, the compound of formula (AA) can be obtained by the process as defined hereinabove.

In some embodiments, in the compounds of Formula (A), (AA), (B) and (BB), n is an integer from 1 to 3, preferably 1 or 2, most preferably 1.

In some embodiments, in the compound of Formula (C), m is an integer from 1 to 3. In a preferred embodiment, m is 2.

In some embodiments, in the compound of Formula (C), LG can be an O-fluorophenyl group such as OPhF₅ or OPhF₄(para SO₃Na), or an O—(N-succinimidyl) group. In a preferred embodiment, LG in the compound of Formula (C) is an O—(N-succinimidyl) group.

In some embodiments, the reaction of the compound of Formula (C) with the compound of Formula (A) to obtain the compound of Formula (B) or with the compound of Formula (AA) to obtain the compound of Formula (BB) is performed in the presence of a photoinitiator. In some embodiments, the photoinitiator can be selected from the group consisting of a free radical-generating azo compound, lithium phenyl-2,4,6-trimethylbenzoylphosphinate (LAP), metals or metal ions-based photoinitiator, peroxides, ammonium persulfate, 2,2-dimethoxy-2-phenylacetophenone (DMPA), and any combination thereof.

In some embodiments, when the photoinitiator is a free radical-generating azo compound, it can be azobisisobutyronitrile (AIBN); 2,2′-azobis(2-methylpropionitrile); 4,4′-azobis(4-cyanopentanoic acid) (ACVA); 1,1′-azobis(cyanocyclohexane) (ACHN); diazenedicarboxylic acid bis(N,N-dimethylamide) (TMAD); azodicarboxylic acid dipiperidide (ADD); 2,2′-azobis[2-(2-imidazolin-2-yl)propane]dihydrochloride; 2,2′-azobis(2-amidinopropane) dihydrochloride (AAPH); 2,2′-azobis(2-methylpropionitrile); 4,4′-(diazene-1,2-diyl)bis(4-cyanopentanoic acid); 2,2′-azodi(2-methylbutyronitrile); or any combination thereof.

In some embodiments, when the photoinitiator is a peroxide, the peroxide can include tert-butyl peroxyisobutyrate, tert-butyl hydroperoxide, benzoyl peroxide or any combination thereof.

In some embodiments, the reaction of the compound of Formula (C) with the compound of Formula (A) or (AA) can be performed in water or in an organic solvent. In some embodiments, the solvent can be selected depending on the solubility of the photoinitiator in this solvent. In some embodiments, one can use a mixture of different solvents if required to enhance the photoinitiator solubility. In some embodiments, the solvent used for performing the reaction of the compound of Formula (C) with the compound of Formula (A) or (AA) can be selected from water, dioxane, acetonitrile, tetrahydrofuran (THF), diisopropyl ether, isopropanol, chlorobenzene, methyl-tert-butyl ether, methanol, ethanol, tert-butanol, chloroform and any combination thereof.

In some embodiments, the reaction of the compound of Formula (C) with the compound of Formula (A) to obtain the compound of Formula (B) or with the compound of Formula (AA) to obtain the compound of Formula (BB) can be performed at a temperature ranging from about 40° C. to about 110° C. In some embodiments, the reaction of the compound of Formula (C) with the compound of Formula (A) or (AA) can be performed for a period of about 1 hour to about 10 hours.

In some embodiments, the reaction of the compound of Formula (C) with the compound of Formula (A) to obtain the compound of Formula (B) or with the compound of Formula (AA) to obtain the compound of Formula (BB) can be performed under ultraviolet light irradiation. In some embodiments, the ultraviolet light irradiation can be short-wave, medium-wave or long-wave ultraviolet light irradiation. In some embodiments, the reaction can even be performed under ultraviolet light irradiation in the presence of a photoinitiator. In such embodiments, the photoinitiator can preferably be selected from 2,2′-azidobis[2-imidazolin-2-yl)propane]dihydrochloride; 2,2-dimethoxy-2-phenylacetophenone (DMPA); and combination thereof. In some embodiments, when the reaction to obtain the compounds of Formula (B) or (BB) is performed under ultraviolet light irradiation, optionally in the presence of a photoinitiator, one can perform the reaction in an alcoholic solvent such as methanol, ethanol, isopropanol or any combination thereof. In some embodiments, the synthesis of the compounds of Formula (B) or (BB) under ultraviolet light irradiation can be performed at a temperature ranging from about 20° C. to about 30° C. In some embodiments, the irradiation can be performed for a period of about 1 hour to about 24 hours.

In some embodiments, the reaction of the compound of Formula (C) with the compound of Formula (A) to obtain the compound of Formula (B) or with the compound of Formula (AA) to obtain the compound of Formula (BB) can be performed under visible light in the presence of a visible light absorbing transition metal photocatalyst. In some embodiments, the visible light absorbing transition metal photocatalyst can be a ruthenium polypyridyl complex such as Ru(bpz)₃(PF₆)₂ and the light source can include blue LED lights. In some embodiments, the reaction that is performed in the presence of a visible light absorbing transition metal photocatalyst, can be performed in a solvent such as acetonitrile at a temperature ranging from about 20° C. to about 30° C.

In some embodiments, the compounds of Formula (B) or (BB) can be isolated using conventional methods. In some embodiments, the reaction mixture comprising compounds of Formula (B) or (BB) can be treated to remove the solvent and any unreacted compound of Formula (C) to recover the compounds of Formula (B) or (BB). Then, the compounds of Formula (B) or (BB) can be directly used in crude form, i.e., without further purification, for conjugation with a carrier material bearing at least one amino group, as will be further detailed below.

### Process Reaction 6a and 6b

In some embodiments, there is also provided a process for preparing a glycoconjugate of Formula (I) or (II)

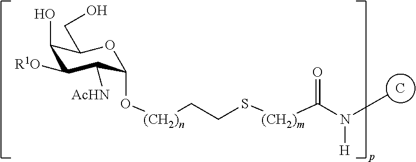

(I): R¹═H
(II): R¹═

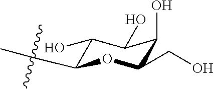

where n is an integer from 1 to 5, m is an integer from 1 to 5, p is an integer from 1 to 50 (e.g., representing the total number of free amino groups on the carrier material available for conjugation) and \textcircled{C} is a carrier material bearing at least one free amino group. The process comprises conjugating the carrier material with a compound of Formula (B) prepared by the process as defined herein to form the glycoconjugate of Formula (I) or with a compound of Formula (BB) prepared by the process as defined herein to form the glycoconjugate of Formula (I).

In some embodiments, the carrier material contains at least one amino group available for conjugation, i.e., at least one amino group capable of reacting with the activated ester group (CO)LG of the compounds of Formula (B) or (BB) for forming the

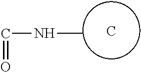

bond in the glyconjugate of Formula (I) or (II). In some embodiments, the carrier material can include from 1 to 50 amino groups allowing the formation of from 1 to 50

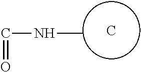

bonds in the glyconjugate of Formula (I) or (II).

In some embodiments, in the compounds of Formula (I) and (II), n is an integer from 1 to 3, preferably 1 or 2, most preferably 1.

In some embodiments, in the compounds of Formula (I) and (II), m is an integer from 1 to 3. In a preferred embodiment, m is 2.

In some embodiments, the conjugation between the compounds of Formula (B) or (BB) and the carrier material can involve any conventional conjugation method.

In some embodiments, the carrier material can comprise a protein, polypeptide or peptide. In other embodiments, the carrier material may comprise a sensor chip, microassay or beads, to name a few examples, which bear free amino groups capable of conjugation with the compounds of Formula (B) or (BB).

In some embodiments, the conjugation reaction between the protein, peptide or polypeptide and the compounds of Formula (B) or (BB) may advantageously minimize or avoid carrier protein or peptide self-crosslinks between the side chains of aspartate/glutamate residues and ε-lysine amines present in the carrier protein or peptide itself.

In some embodiments, the conjugation reactions described herein enable the number of carbohydrate antigens conjugated to the carrier protein or peptide to be controlled by the efficacy and/or stoichiometry of the reactants (e.g., the molar ratio of the carrier protein or peptide to the compounds of Formula (B) or (BB)). In some embodiments, the conjugation reactions described herein may comprise reacting between 1 to 500, 1 to 400, 1 to 300, 1 to 200, 5 to 500, 5 to 400, 5 to 300, or 5 to 200 molar equivalents of the compound of Formula (B) or (BB) per carrier protein or peptide.

In some embodiments, the carrier proteins or peptides described herein comprise one or more free amine groups. As used herein, “free amine” or “free amine group” refers to carrier proteins or peptides having one or more amino groups that are available for chemical modification and/or conjugation (e.g., to a carbohydrate antigen as described herein, such as solvent accessible lysine residues that tend to be exposed on the periphery of the carrier protein). In some embodiments, it may be advantageous to avoid having too many multiple carbohydrate antigens conjugated to adjacent positions on the carrier proteins. In some embodiments, the carrier protein or peptide may preferably lack a lysine-rich domain (e.g., a segment of at least 4, 5, 6, 7, 8, 9, or 10 consecutive amino acids comprising at least 50% of lysine residues).

In some embodiments, the carrier protein may comprise at least 1, 2, 3, 4, 5, 6, 7, 8, 9, 10, 11, 12, 13, 14, 15, 16, 17 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 total lysine residues. In some embodiments, the carrier protein may comprise 1, 2, 3, 4, 5, 6, 7, 8, 9, 10, 11, 12, 13, 14, 15, 16, 17 18, 19, 20, 21, 22, 23, 24, 25, 26, 27, 28, 29, or 30 total free amine residues.

In some embodiments, the carrier protein or peptide comprises one or more lysine residues having the one or more free amine groups, or optionally is engineered to add one or more further lysine residues, for example at the amino terminus, the carboxy terminus, or a solvent-accessible position of the carrier protein or peptide. In some embodiments, the carrier protein comprises a T cell epitope, and/or induces a cell-mediated immune response in the subject. In some embodiments, the carrier protein or peptide comprises a B cell epitope, and/or induces a humoral immune response in the subject. In some embodiments, the carrier protein comprises both a B cell epitope and a T cell epitope, and/or induces both a humoral and a cell-mediated immune response in the subject.

Preferably, the carrier protein described herein may be a protein that has already received regulatory (e.g., FDA) approval for administration to human subjects (e.g., in approved vaccines). In some embodiments, the carrier protein is, is from, or comprises: Tetanus Toxoid (TT), Diphtheria Toxoid (DT), cross-reacting material 197 (CRM197), Meningococcal Outer Membrane Protein Complex (OMPC), H. Influenzae Protein D (HiD), a virus-like particle (VLP), a cytokine, an immunogenic peptide such as Tetanus Toxin 831-844 (SEQ ID NO: 1 or 2), Tetanus Toxin 830-843 (SEQ ID NO: 5), albumin (such as bovine serum albumin or human serum albumin), keyhole limpet hemocyanin (KLH), or an immunogenic fragment thereof.

In some embodiments, the carrier protein or peptide is exogenous to the subject to be administered, which preferably has no (close) ortholog in the subject. In the context of human vaccine production, a carrier protein described herein refers to a “carrier protein suitable for human use” or simply “suitable carrier protein”, which means a carrier protein that is antigenically distinct from human proteins such that the carrier protein would not be considered as a “self-antigen” in humans. The use of carrier proteins that are too antigenically similar to corresponding human proteins may result in the carrier protein being considered as a “self-antigen”, which may not be ideal in human vaccines. For example, glycoconjugate immunogens consisting of TF antigen randomly conjugated to the F-amino groups of lysine residues of bovine serum albumin (BSA) have been previously described and characterized (e.g., Demian et al., 2014; Rittenhouse-Diakun et al., 1998; Heimburg et al., 2006; Tati et al., 2017). However, not only was the level of carbohydrate on the 59 lysine residues of BSA random and inefficient (no more than 4 to 6 TF antigens were conjugated per BSA molecule), BSA would not be suitable as a carrier protein in human vaccines because it is too antigenically similar to human albumin. In some embodiments, the carrier protein is not albumin (e.g., bovine serum albumin).

In some embodiments, the glycoconjugates of Formula (I) or (II) described herein may be glycoconjugate immunogens, wherein the carrier protein or peptide is immunogenic when administered to a subject, and conjugation of the carbohydrate antigen to the carrier protein or peptide via the thio-linker increases the immunogenicity of the carbohydrate antigen upon administration to the subject as compared to a corresponding administration of the unconjugated carbohydrate antigen.

In some embodiments, the carbohydrate antigen or carbohydrate antigen, following coupling to the carrier protein or peptide, is not cleavable from the carrier protein or peptide by an endogenous enzyme of the subject.

In some embodiments, the carrier proteins or peptides described herein may comprise a T cell epitope, and/or induce a cell-mediated immune response in the subject upon administration.

In some embodiments, the synthetic glycoconjugate of Formula (I) or (II) described herein may induce a cell-mediated immune response to the carbohydrate antigen upon administration to the subject.

In a further aspect, described herein is a method for producing a glycoconjugate vaccine or an immune response-triggering composition. The method may comprise formulating a glycoconjugate of Formula (I) or (II) as prepared by a method as described herein with a pharmaceutically acceptable excipient, and/or an adjuvant. In some embodiments, the adjuvant is or comprises: an inorganic compound, a mineral oil, a microbial derivative, a plant derivative, a cytokine, squalene, alum, aluminum hydroxide, aluminum phosphate, calcium phosphate hydroxide, a toll-like receptor agonist, an immunostimulatory polynucleotide (e.g., CPG), an immunostimulatory lipid, Freund's adjuvant, RIBI's adjuvant, QS-21, muramyl dipeptide, TiterMax™, Steviune™, Stimune™, or any combination thereof.

Vaccine compositions can be administered in dosages and by techniques well known to those skilled in the medical or veterinary arts, taking into consideration such factors as the age, sex, weight, species and condition of the recipient animal, and the route of administration. The route of administration can be percutaneous, via mucosal administration (e.g., oral, nasal, ocular) or via a parenteral route (e.g., intradermal, intramuscular, subcutaneous). Vaccine compositions can be administered alone, or can be co-administered or sequentially administered with other treatments or therapies. Forms of administration may include suspensions and preparations for parenteral, subcutaneous, intradermal or intramuscular administration (e.g., injectable administration) such as sterile suspensions or emulsions. Vaccines may be administered as a spray or mixed in food and/or water or delivered in admixture with a suitable carrier, diluent, or excipient such as sterile water, physiological saline, glucose, or the like. The compositions can contain auxiliary substances such as wetting or emulsifying agents, pH buffering agents, adjuvants, gelling or viscosity enhancing additives, preservatives, flavoring agents, colors, and the like, depending upon the route of administration and the preparation desired. Standard pharmaceutical texts, such as “Remington's Pharmaceutical Sciences,” 1990 may be consulted to prepare suitable preparations, without undue experimentation.

In a further aspect, described herein is a glycoconjugate vaccine or an adaptive immune response-triggering composition comprising a glycoconjugate of Formula (I) or (II) prepared by the method described herein and a pharmaceutically acceptable excipient and/or adjuvant as described herein. In embodiments, the glycoconjugate vaccine may be a prophylactic vaccine or a therapeutic vaccine. In embodiments, the vaccine compositions described herein may comprise one or more TACAs and the vaccine composition may be an anti-cancer vaccine against a cancer expressing the TACA. In embodiments, the cancer may be B-cell lymphoma, breast cancer, colon cancer, non-small cell lung cancer, melanoma, neuroblastoma, ovary, prostate, sarcoma, small cell lung cancer, or stomach cancer.

In some aspects, described herein is a method of immunizing, vaccinating, or treating a subject comprising administering to the subject a glycoconjugate of Formula (I) or (II) produced by a method as described herein, a synthetic glycoconjugate of Formula (I) or (II) as described herein, a glycoconjugate vaccine or an adaptive immune response-triggering composition produced by a method as described herein, or a glycoconjugate vaccine as described herein.

In some embodiments, described herein is a glycoconjugate of Formula (I) or (II) produced by a method as described herein, a synthetic glycoconjugate of Formula (I) or (II) as described herein, a glycoconjugate vaccine or an adaptive immune response-triggering composition produced by a method as described herein, or a glycoconjugate vaccine as described herein, for use in immunizing, vaccinating, or treating a subject having a disease, or for detecting the presence of an antibody that specifically binds to the glycoconjugate of Formula (I) or (II) or for detecting said immunization, vaccination, or treatment (e.g., in a biological sample from the subject).

In some embodiments, described herein is a glycoconjugate of Formula (I) or (II) produced by a method as described herein, a synthetic glycoconjugate as described herein, or an adaptive immune response-triggering composition produced by a method as described herein, for the manufacture of a vaccine for immunizing or treating a subject having a disease, or for detecting the presence of an antibody that specifically binds to the glycoconjugate of Formula (I) or (II) or for detecting said immunization or treatment (e.g., in a biological sample from the subject).

In some embodiments, described herein is a glycoconjugate of Formula (I) or (II) produced by a method as described herein, a synthetic glycoconjugate of Formula (I) or (II) as described herein, a glycoconjugate vaccine or an adaptive immune response-triggering composition produced by a method as described herein, or a glycoconjugate vaccine as described herein, for use in the treatment of a subject having a disease associated with increased expression of said carbohydrate antigen.

In some embodiments, described herein is a glycoconjugate of Formula (I) or (II) produced by a method as described herein, a synthetic glycoconjugate of Formula (I) or (II) as described herein, a glycoconjugate vaccine or an adaptive immune response-triggering composition produced by a method as described herein, or a glycoconjugate vaccine as described herein, for detecting or screening for the presence of an antibody that specifically binds to the carbohydrate antigen or a tumor-circulating cell comprising the carbohydrate antigen, or for detecting the presence of antibodies resulting from an immunization or vaccination with the carbohydrate antigen. In some embodiments, the detection or screening may be performed via any suitable detection method such as an immunosorbent assay, ELISA, microarray, or immunoblot analysis.

In further aspects, described herein is a method of treating a subject comprising administering a glycoconjugate of Formula (I) or (II) produced by a method as described herein, to generate an immune response in said subject to a carbohydrate antigen, and optionally screening a biological sample from said subject for the presence of antibodies that specifically binds to the carbohydrate antigen.

In a further aspect, described herein is a glycoconjugate of Formula (I) or (II) for use as therapeutic and/or diagnostic tools relating to the SARS-CoV-2. More particularly, described herein is a glycoconjugate of Formula (I) or (II) for use in immunizing a subject against SARS-CoV-2, for use in triggering the production of anti-SARS-CoV-2 antibodies in a subject, for use in inducing a cell-mediated immune response in a subject against SARS-CoV-2, or any combination thereof. Also described herein is a glycoconjugate of Formula (I) or (II) for use in detection/diagnostic tools relating to SARS-CoV-2. For example, described herein is a glycoconjugate of Formula (I) or (II) for use in detecting the presence of anti-SARS-CoV-2 antibodies in a sample from a subject. In this regard, results demonstrating the presence of carbohydrate antigens (e.g., TF, Tn, and sialylated variants thereof) on the S protein of SARS-CoV-2, as well as their accessibility to binding by anti-carbohydrate ligands is shown in PCT/CA2020/051253.

As used herein, the expression “anti-SARS-CoV-2 antibodies” refers to antibodies that are able to bind to antigens (e.g., carbohydrate antigens) in their native conformations, such as expressed on native recombinant proteins and/or present on assembled virion particles. In contrast, antibodies that bind only to denatured antigens (e.g., under denaturing conditions such as following SDS-PAGE) but not to the same antigens in their native conformations are excluded from the expression “anti-SARS-CoV-2 antibodies”. In some embodiments, the glycoconjugates of Formula (I) or (II) or vaccine described herein may induce the production of antibodies having neutralizing activity. As used herein, the expression “neutralizing activity” refers to ligands (e.g., antibodies) that bind to SARS-CoV-2 virion particles and inhibit their ability to infect susceptible host cells.

In some embodiments, the glycoconjugates of Formula (I) or (II) described herein may comprise carbohydrate antigens conjugated to a suitable carrier material (e.g., a carrier protein or peptide, or a non-proteinaceous polymeric material), wherein the carbohydrate antigens comprise or consists of TF antigen, Tn antigen, or any combination thereof.

In some embodiments, the carbohydrate antigens described herein may be conjugated to a carrier material that comprises a B cell epitope or T cell epitope, for example depending on whether triggering a humoral and cell-mediated immune response is desired. In some embodiments, the carbohydrate antigens may be covalently conjugated to the SARS-CoV-2 S protein fragment of SEQ ID NO: 3 or 4, such as at positions 4 and/or 6 of SEQ ID NO: 3 or at positions 323, 325, and/or 678 of SEQ ID NO: 4. In particular, position 678 of SEQ ID NO: 4 (which is close to the furin cleavage site of the spike protein at R682) has been reported to be O-glycosylated by core-1 and core-2 structures.

In some embodiments, the carrier protein or peptide may comprise an immunogenic fragment of the SARS-CoV-2 S protein sequence of SEQ ID NO: 4, the fragment comprising one or more carbohydrate antigens conjugated to position 323, 325, and/or 678 of SEQ ID NO: 4. In some embodiments, the carbohydrate antigens may be covalently conjugated to a variant of the SARS-CoV-2 S protein fragment of SEQ ID NO: 3, for example a variant wherein the residues at positions 4 and/or 6 may be replaced with lysine and/or cysteine residues, which may facilitate chemical conjugation to the carbohydrate antigens. In some embodiments, the carrier protein or peptide may comprise an immunogenic fragment of a variant of the SARS-CoV-2 S protein sequence of SEQ ID NO: 4 having a lysine or cysteine at positions 323, 325, and/or 678, the fragment comprising one or more carbohydrate antigens conjugated to the lysine or cysteine residues at position 323, 325, and/or 678 of SEQ ID NO: 4. In the case of lysine residues, the carbohydrate antigens may be conjugated to the carrier protein via conjugation methods described herein. In the case of cysteine residues, the carbohydrate antigens may be conjugated to the carrier protein via conjugation methods described. Thus, in some embodiments, the carrier material described herein may comprise or consist of the peptide of SEQ ID NO: 3, or to a variant of the peptide of SEQ ID NO: 3 comprising a cysteine or lysine at positions 4 and/or 6. In some embodiments, the peptide or peptide variant of SEQ ID NO: 3 may be comprised in (e.g., recombinantly engineered into the amino acid sequence) or may be fused to (e.g., as a fusion protein) the carrier material.

In some embodiments, the carrier material is, is from, or comprises: Tetanus Toxoid (TT), Diphtheria Toxoid (DT), cross-reacting material 197 (CRM197), Meningococcal Outer Membrane Protein Complex (OMPC), H. Influenzae Protein D (HiD), a virus-like particle (VLP), a cytokine, an immunogenic peptide such as Tetanus Toxin 831-844 (SEQ ID NO: 1 or 2), Tetanus Toxin 830-843 (SEQ ID NO: 5), albumin (such as bovine serum albumin or human serum albumin), keyhole limpet hemocyanin (KLH), or an immunogenic fragment thereof.

In some aspects, described herein is a SARS-CoV-2 or COVID-19 vaccine or adaptive immune response-inducing composition comprising one or more glycoconjugates of Formula (I) or (II) as defined herein, and a pharmaceutically acceptable excipient and/or an adjuvant. The glycoconjugates generally comprise one or more carbohydrate antigens expressed on SARS-CoV-2 virions, such as for example carbohydrate antigens expressed on the S (or S1) protein of SARS-CoV-2. The carbohydrate antigens suitable for a SARS-CoV-2 vaccine as described herein are carbohydrate antigens that are aberrant glycosylation patterns—i.e., those not expressed on normal or healthy cells and tissues of a subject—in order to reduce the risk of triggering an auto-immune response in the subject being administered the vaccine.

In some embodiments, the glycoconjugates of Formula (I) or (II) described herein or the SARS-CoV-2 vaccines described herein, induce the production of antibodies that bind to SARS-CoV-2 virion particles, and preferably have neutralizing activity (e.g., inhibit the ability of SARS-CoV-2 virion particles from infecting susceptible host cells).

In another aspect, the present description also relates to a compound of Formula (AA_{PG})

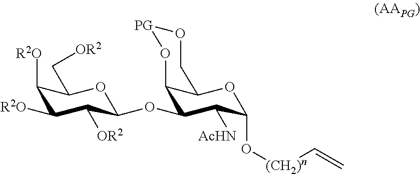

where n is an integer from 1 to 5, R² is Ac and PG is a divalent protecting group selected from PhCH, Me₂C, O═C, Me₂Si and 2,6-bis(trifluoromethyl)phenyl-B.

In some embodiments, the divalent PG protecting group is PhCH.

In some embodiments, in the Formula (AA_{PG}), n is an integer from 1 to 3. In a preferred 20 embodiment, n is 1 or 2. In another embodiment, n is 1.

In some embodiments, the compound of Formula (AA_{PG}) can be used as an intermediate in the production of the glycoconjugates of Formula (I) or (II) described herein. In some embodiments, the compound of Formula (AA_{PG}) can itself be prepared by the process described herein.

In some embodiments, the compound (AA_{PG}) has the following formula:

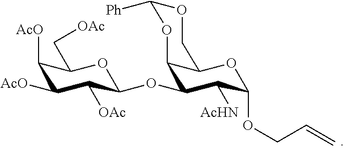

In a further aspect, the present description also relates to a compound of formula:

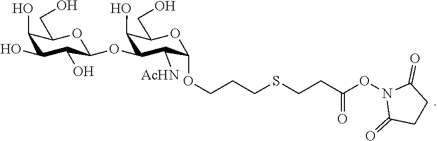

### Items

Described herein are one or more of the following items.
1. A process for synthesizing a reactive carbohydrate of Formula (A)

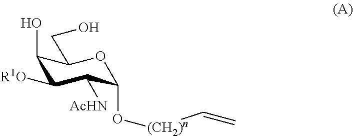

where R¹ is H and n is an integer from 1 to 5,
comprising reacting, under heating, a compound of Formula (A₀)

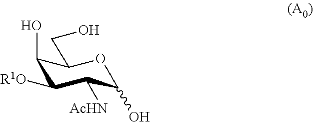

with HO—(CH₂)ₙ—CH═CH₂ in the presence of an acid capable of liberating a proton, resulting in a reaction mixture comprising the compound of Formula (A), and
cooling the reaction mixture to obtain a cooled mixture comprising the compound of Formula (A).
2. The process of item 1, wherein the acid comprises acetyl chloride, acetic acid, an acidic cation exchange resin, camphorsulfonic acid, p-toluenesulfonic acid monohydrate, HCl or any mixture thereof.
3. The process of item 1 or 2, wherein the acid comprises acetyl chloride.
4. The process of any one of items 1 to 3, wherein reacting the compound of Formula (A₀) comprises adding the compound of Formula (A₀) to a solution comprising HO—(CH₂)ₙ—CH═CH₂ and acetyl chloride at a mixing temperature from about 0° C. to about 25° C. resulting in an intermediate reaction mixture at low temperature comprising the compound of Formula (A₀), HO—(CH₂)ₙ—CH═CH₂ and HCl.
5. The process of item 4, wherein the mixing temperature is about 0° C., or about 0° C. to about 10° C., or about 0° C. to about 20° C., or about 10° C. to about 20° C., about 10° C. to about 25° C.
6. The process of item 4 or 5, wherein the solution comprising HO—(CH₂)ₙ—CH═CH₂ and acetyl chloride is prepared at a temperature of about 0° C. under inert atmosphere.
7. The process of any one of items 4 to 6, wherein reacting the compound of Formula (A₀) comprises contacting the intermediate reaction mixture with a heat source at a temperature of from about 40° C. to about 80° C.
8. The process of any one of items 1 to 7, wherein the heating is performed at a reaction temperature from about 40° C. to about 80° C. for a reaction time of from about 30 minutes to 5 hours.
9. The process of item 8, wherein the reaction temperature is from about 60° C. to about 80° C.
10. The process of item 8 or 9, wherein the reaction temperature is about 70° C.
11. The process of any one of items 8 to 10, wherein the reaction time is from about 30 min to about 1 hour.
12. The process of any one of items 8 to 11, wherein the reaction time is from about 30 min to about 45 min.
13. The process of any one of items 8 to 12, wherein the reaction time is about 30 min.
14. The process of item 1 or 2, wherein the acid comprises acetic acid, an acidic cation exchange resin, camphorsulfonic acid, p-toluenesulfonic acid monohydrate, HCl or any mixture thereof.
15. The process of item 14, wherein reacting the compound of Formula (A₀) with HO—(CH₂)ₙ—CH═CH₂ comprises heating a solution comprising the compound of Formula (A₀), HO—(CH₂)ₙ—CH═CH₂ and the acid.
16. The process of item 15, wherein the solution further comprises a solvent selected from the group consisting of dichoromethane, chloroform, 1,3-dioxolane, diethoxymethane, dimethoxymethane, 2,5,7,10-tetraoxaundecane, dipropoxymethane and any combination thereof.
17. The process of item 15 or 16, wherein the reaction is performed at a temperature of from about 30° C. to about 70° C.
18. The process of any one of items 15 to 17, wherein the reaction is performed for a reaction time of about 0.5 hour to about 15 hours, or from about 0.5 hour to about 10 hours, or from about 0.5 hour to about 5 hours, or from about 0.5 hour to about 2 hours.
19. The process of any one of items 1 to 18, further comprising neutralizing the cooled mixture.
20. The process of any one of items 1 to 19, further comprising isolating the compound of Formula (A).
21. A process for synthesizing a reactive carbohydrate of Formula (AA_{PG})

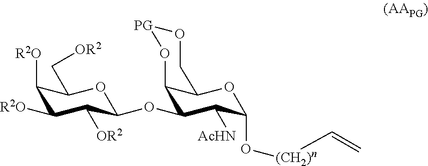

where R² is a monovalent protecting group, PG is a divalent protecting group and n is an integer from 1 to 5,
comprising reacting, in the presence of a metal-containing zeolite, a compound of Formula (A_{PG})

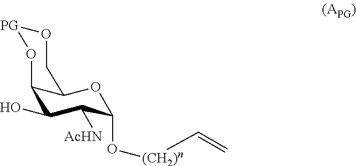

with a compound of Formula (A₁)

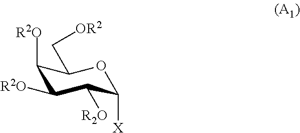

where X is —I, —Br, —Cl, —CN, —OTf (Triflate), —OMs (Mesylate), —OTs (Tosylate), methylsulfate, or trichloroacetimidate.
22. The process of item 21, wherein the metal in the metal-containing zeolite comprises Ag, Al, Cd, Co, Cu, Fe, Ga, In, Mo, Pd, Pt, Sn, Sb, V, Zr or any mixture thereof.
23. The process of item 21, wherein the metal-containing zeolite comprises a zeolite selected from the group consisting of Sn-Beta, Zr-Beta, Al-Beta(OH), Al-Beta(F), Pt@MCM-22, K-PtSn/MFI, 0.3Pt/0.5Sn—Si-Beta, Pt/Sn 2.0-Beta, 0.5CoSi-Beta, V-Beta, H—[Fe]ZSM-5, Fe-BEA, Ga-Beta, Ga-Beta-200, Mo/HZSM-5, or any mixture thereof.
24. The process of item 21, wherein the metal-containing zeolite comprises a silver-containing zeolite.
25. The process of any one of items 21 to 24, wherein the reaction is performed in an organic solvent selected from the group consisting of toluene, dichorometane-toluene mixture, DMSO-toluene mixture, and N-methylpyrrolidone-toluene mixture.
26. The process of any one of items 21 to 25, wherein the reaction is performed in toluene.
27. The process of any one of items 21 to 26, wherein the reaction is performed in the presence of a molecular sieve.
28. The process of any one of items 21 to 27, wherein the reaction is performed in the presence of a molecular sieve of type 3 Å, type 4 Å, type 5 Å, type 13X or any mixture thereof.
29. The process of any one of items 21 to 28, wherein the reaction is performed at a temperature of from about 20° C. to about 80° C.
30. The process of any one of items 21 to 29, wherein the reaction is performed at a temperature of from about 20° C. to about 50° C.
31. The process of any one of items 21 to 30, wherein the reaction is performed at a temperature of from about 20° C. to about 30° C.
32. The process of any one of items 21 to 31, wherein the reaction is performed for about 5 hours to about 50 hours.
33. The process of any one of items 21 to 32, wherein the reaction is performed for about 5 hours to about 30 hours.
34. The process of any one of items 21 to 31, wherein the reaction is performed at about 60 to about 80° C. for about 15 hours to about 20 hours.
35. The process of any one of items 21 to 31, wherein the reaction is performed at about 20° C. to about 25° C. for about 20 hours to about 30 hours.
36. The process of any one of items 21 to 35, wherein R² is selected from the group consisting of Ac, Bz, allyl, benzoate, methoxymethyl (MOM), tetrahydropropyranyl (THP), t-butyl, pivalate, t-butyldimethylsilyl (TBDMS) and t-butyldiphenylsilyl (TBDPS).
37. The process of any one of items 21 to 36, wherein R² is Ac or Bz.
38. The process of any one of items 21 to 37, wherein PG is selected from the group consisting of PhCH, Me₂C, O═C, Me₂Si and 2,6-bis(trifluoromethyl)phenyl-B.
39. The process of any one of items 21 to 38, wherein PG is PhCH.
40. The process of any one of items 21 to 39, wherein X is Br.
41. The process of any one of items 21 to 40, wherein the reaction is performed under inert atmosphere.
42. The process of any one of items 21 to 41, further comprising isolating the compound of Formula (AA_{PG}).
43. The process of any one of items 21 to 42, wherein the compound of Formula (A_{PG}) is formed by protecting a compound of Formula (A)

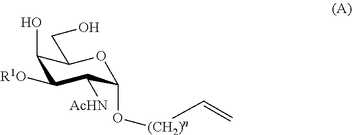

where R¹ is H, with the protecting group PG.
44. The process of item 43, wherein protecting the compound of Formula (A) is performed by reaction with a compound selected from the group consisting of PhCH(OMe)₂, triphosgene, Me₂SiCl₂, Me₂C(OMe)₂, 2-methoxypropene and 2,6-bis(trifluoromethyl)phenylboronic ester.
45. The process of item 43 or 44, wherein protecting the compound of Formula (A) is performed in an organic solvent selected from the group consisting of DMF, Dimethyl acetamide (DMAC), DMSO, THF, Dioxane, 1-3 Dioxolane, acetonitrile, 2,5-dimethyl tetrahydrofuran (DMTHF), Gamma-vaterolactone (GVL), Dihydrolevoglucoserone (Cyrene), methyl levulinate (ML), Ethyl levulinate (EL), Ethyl levulinate propyleneglycol ketal (ELPK), Dimethyl glutarate (DMG), Dimethylpropylene urea (DMPU), Poly(propyleneglycol) (PPG), Glycofurol (THFP), 1-Ethyl-3-methylimidazolium acetate ([emim][OAc]) and any combination thereof.
46. The process of any one of items 43 to 45, wherein the compound of Formula (A) is obtained by the process of any one of items 1 to 20.
47. A process for synthesizing a reactive carbohydrate of Formula (AA)

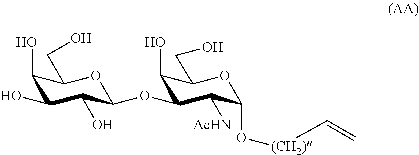

where n is an integer from 1 to 5,
comprising deprotecting the compound of Formula (AA_{PG}) obtained by the process of any one of items 21 to 46.
48. The process of item 47, wherein deprotecting comprises reacting the compound of Formula (AA_{PG}) with a basic solution to form an intermediate product, and then treating the intermediate product with an acidic solution.
49. The process of item 48, wherein the basic solution comprises a base is selected from the group consisting of a methoxide, ethoxide and 2-methylpropan-2-olate, preferably the base is sodium methoxide.
50. The process of item 48 or 49, wherein the basic solution comprises a solvent selected from the group consisting of methanol, ethanol and isopropanol, preferably the solvent is methanol.
51. The process of any one of items 48 to 50, wherein the acidic solution comprises an acetic acid-water mixture or an ethanolic acid-water mixture, preferably the acidic solution is an aqueous acetic acid solution.
52. The process of any one of items 48 to 51, wherein the reaction with the basic solution is performed at a temperature of from about 0° C. to about 50° C., preferably at room temperature.
53. The process any one of items 48 to 52, wherein the reaction with the basic solution is performed for a period of about 0.5 hour to about 5 hours, preferably about 1 hour to about 2 hours.
54. The process of any one of items 48 to 53, wherein treating with the acidic solution is performed at a temperature of from about 50° C. to about 70° C., preferably at about 60° C.
55. The process of any one of items 48 to 54, wherein treating with the acidic solution is performed for a period of about 1 hour to about 5 hours, preferably about 1 hour to about 2 hours.
56. The process of any one of items 48 to 55, further comprising neutralizing the basic solution after reacting with the compound of Formula (AA_{PG}) and evaporating any liquid to afford the intermediate product in crude form.
57. The process of any one of items 48 to 56, wherein the intermediate product is crude when treated with the acidic solution.
58. A process for synthesizing a reactive carbohydrate of Formula (AA)

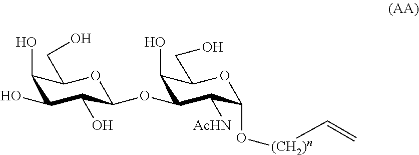

comprising a deprotection a compound of Formula (AA_{PG})

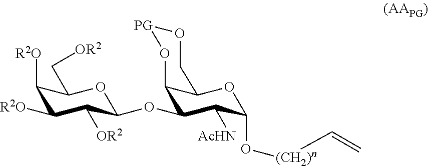

where n is an integer from 1 to 5, R² is a monovalent protecting group, and PG is a divalent protecting group,
the deprotection comprising reacting the compound of Formula (AA_{PG}) with a basic solution to form an intermediate crude product, and then treating the intermediate crude product with an acidic solution.
59. The process of item 58, wherein the basic solution comprises a base selected from the group consisting of a methoxide, ethoxide and 2-methylpropan-2-olate, preferably the base is sodium methoxide.
60. The process of item 58 or 59, wherein the basic solution comprises a solvent selected from the group consisting of methanol, ethanol and isopropanol, preferably the solvent is methanol.
61. The process of any one of items 58 to 60, wherein the acidic solution comprises an acetic acid-water mixture or an ethanolic acid-water mixture, preferably the acidic solution is an aqueous acetic acid solution.
62. The process any one of items 58 to 61, wherein the reaction with the basic solution is performed at a temperature of from about 0° C. to about 50° C., preferably at room temperature.
63. The process any one of items 58 to 62, wherein the reaction with the basic solution is performed for a period of about 0.5 hour to about 5 hours, preferably about 1 hour to about 2 hours.
64. The process of any one of items 58 to 63, wherein treating with the acidic solution is performed at a temperature of from about 50° C. to about 70° C., preferably at about 60° C.
65. The process of any one of items 58 to 64, wherein treating with the acidic solution is performed for a period of about 1 hour to about 5 hours, preferably about 1 hour to about 2 hours.
66. The process of any one of items 58 to 65, further comprising neutralizing the basic solution after reacting with the compound of Formula (AA_{PG}) and evaporating any liquid to afford the intermediate crude product.
67. The process of any one of items 58 to 66, wherein R² is Ac or Bz, preferably Bz.
68. The process of any one of items 58 to 67, wherein PG is selected from the group consisting of PhCH, Me₂C, O═C, Me₂Si and 2,6-bis(trifluoromethyl)phenyl-B.
69. The process of any one of items 58 to 68, wherein PG is PhCH.
70. A process for synthesizing a reactive carbohydrate of Formula (B) or Formula (BB)

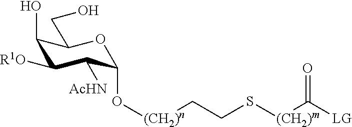

(B): R¹ is H
(BB): R¹ is

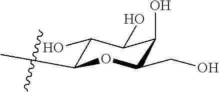

where n is an integer from 1 to 5, m is an integer from 1 to 5 and LG is a leaving group enabling an amido bond formation when the compound of Formula (B) or (BB) is reacted with an amino group,
comprising reacting a compound of Formula (C)

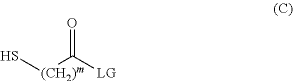

with a compound of Formula (A)

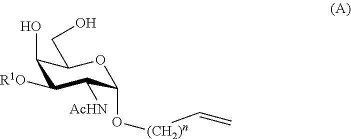

where R¹ is H, to form the compound of Formula (B)
or
with a compound of Formula (AA)

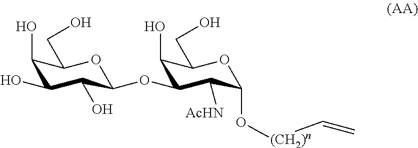

to form the compound of Formula (BB).
71. The process of item 70, wherein reacting the compound of Formula (C) with the compound of Formula (A) or (AA) is performed in the presence of a photoinitiator.
72. The process of item 71, wherein the photoinitiator is selected from the group consisting of a free radical-generating azo compound, lithium phenyl-2,4,6-trimethylbenzoylphosphinate (LAP), metals or metal ions-based photoinitiator, peroxides, ammonium persulfate, 2,2-dimethoxy-2-phenylacetophenone (DMPA), and any combination thereof.
73. The process of item 72, wherein the free radical-generating azo compound is selected from the group consisting of azobisisobutyronitrile (AIBN); 2,2′-azobis(2-methylpropionitrile); 4,4′-azobis(4-cyanopentanoic acid) (ACVA); 1,1′-azobis(cyanocyclohexane) (ACHN); diazenedicarboxylic acid bis(N,N-dimethylamide) (TMAD); azodicarboxylic acid dipiperidide (ADD); 2,2′-azobis[2-(2-imidazolin-2-yl)propane]dihydrochloride; 2,2′-azobis(2-amidinopropane) dihydrochloride (AAPH); 2,2′-azobis(2-methylpropionitrile); 4,4′-(diazene-1,2-diyl)bis(4-cyanopentanoic acid); 2,2′-azodi(2-methylbutyronitrile); and any combination thereof.
74. The process of item 72 or 73, wherein the peroxide is selected from the group consisting of tert-butyl peroxyisobutyrate, tert-butyl hydroperoxide, benzoyl peroxide and any combination thereof.
75. The process of any one of items 70 to 74, wherein reacting the compound of Formula (C) with the compound of Formula (A) or (AA) is performed in water or an organic solvent.
76. The process of any one of items 70 to 75, wherein reacting the compound of Formula (C) with the compound of Formula (A) or (AA) is performed in a solvent selected from the group consisting of water, dioxane, acetonitrile, tetrahydrofuran (THF), diisopropyl ether, isopropanol, chlorobenzene, methyl-tert-butyl ether, methanol, ethanol, tert-butanol, chloroform and any combination thereof.
77. The process of any one of items 70 to 76, wherein reacting the compound of Formula (C) with the compound of Formula (A) or (AA) is performed at a temperature ranging from about 40° C. to about 110° C.
78. The process of any one of items 70 to 77, wherein reacting the compound of Formula (C) with the compound of Formula (A) or (AA) is performed for a period of about 1 hour to about 10 hours.
79. The process of item 70, wherein reacting the compound of Formula (C) with the compound of Formula (A) or (AA) is performed under visible light in the presence of a visible light absorbing transition metal photocatalyst.
80. The process of item 70, wherein reacting the compound of Formula (C) with the compound of Formula (A) or (AA) is performed under ultraviolet light irradiation.
81. The process of item 70, wherein reacting the compound of Formula (C) with the compound of Formula (A) or (AA) is performed under ultraviolet light irradiation in the presence of a photoinitiator.
82. The process of item 81, wherein the photoinitiator is selected from the group consisting of 2,2′-azidobis[2-imidazolin-2-yl)propane]dihydrochloride; 2,2-dimethoxy-2-phenylacetophenone (DMPA); and combination thereof.
83. The process of any one of items 80 to 82, wherein reacting the compound of Formula (C) with the compound of Formula (A) or (AA) is performed in an alcoholic solvent such as methanol, ethanol, isopropanol or any combination thereof.
84. The process of any one of items 80 to 83, wherein reacting the compound of Formula (C) with the compound of Formula (A) or (AA) is performed at a temperature ranging from about 20° C. to about 30° C.
85. The process of any one of items 80 to 84, wherein reacting the compound of Formula (C) with the compound of Formula (A) or (AA) is performed for a period of about 1 hour to about 24 hours.
86. The process of any one of items 80 to 85, wherein the ultraviolet light irradiation is short-wave, medium-wave or long-wave ultraviolet light irradiation.
87. The process of any one of items 70 to 86, wherein m is an integer from 1 to 3.
88. The process of any one of items 70 to 87, wherein m is 2.
89. The process of any one of items 70 to 88, wherein LG is an O-fluorophenyl group such as OPhF₅ or OPhF₄(para SO₃Na), or an O—(N-succinimidyl) group.
90. The process of any one of items 70 to 89, wherein LG is an O—(N-succinimidyl) group.
91. The process of any one of items 70 to 90, wherein the compound of Formula (A) is obtained by the process of any one of items 1 to 20.
92. The process of any one of items 70 to 90, wherein the compound of Formula (AA) is obtained by the process of any one of items 47 to 69.
93. A process for preparing a glycoconjugate of Formula (I) or (II)

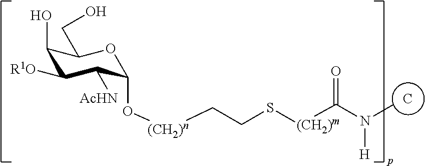

(I) R¹ ═H
(ii): R¹═

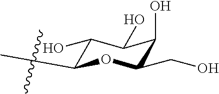

where n is an integer from 1 to 5, m is an integer from 1 to 5, p is an integer from 1 to 50 and

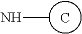

is a carrier material containing at least one amino group available for conjugation, comprising conjugating at least one free amino group of the carrier material with a compound of Formula (B) to form the glycoconjugate of Formula (I) or with a compound of Formula (BB) to form the glycoconjugate of Formula (I), wherein the compounds of Formula (B) and (BB) are obtained by the process of any one of items 70 to 92.
94. The process of item 93, wherein the carrier material comprises a protein, polypeptide or peptide.
95. The process of any one of items 1 to 94, wherein n is an integer from 1 to 3.
96. The process of any one of items 1 to 95, wherein n is 1 or 2.
97. The process of any one of items 1 to 96, wherein n is 1.
98. A compound of Formula (AA_{PG})

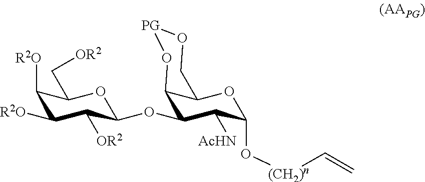

where n is an integer from 1 to 5, R² is Ac and PG is a divalent protecting group selected from PhCH, Me₂C, O═C, Me₂Si and 2,6-bis(trifluoromethyl)phenyl-B.
99. The compound of item 98, wherein PG is PhCH.
100. The compound of item 98 or 99, wherein n is an integer from 1 to 3.
101. The compound of any one of items 98 to 100, wherein n is 1 or 2.
102. The compound of any one of items 98 to 101, wherein n is 1.
103. The compound of item 98, having the formula:

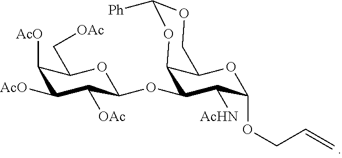

104. A compound of formula:

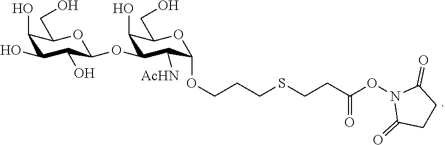

### EXAMPLES

Various reactive carbohydrates were synthesized according to reaction Scheme 2 and as further detailed below. In addition, protein conjugation to form Glyconjugates (I) and (II) was performed and conjugation assessed by ELISA, Western blot, MALDI-TOF and Bradford assay.

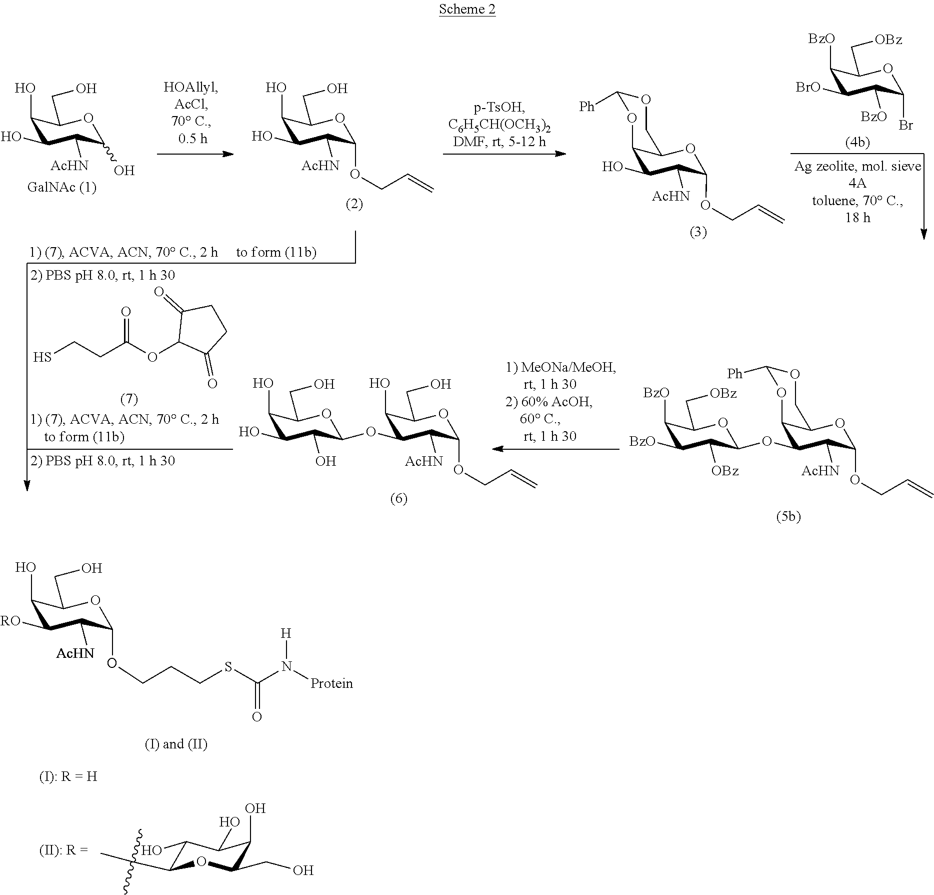

Reactions were carried out under argon atmosphere using commercially available HPLC grade. Commercially available reagents (Sigma Aldrich and Fisher Scientific, Canada) were used without further purification. N-Acetyl-D-galactosamine was provided from Rose Scientific Ltd. Alberta, Canada. Progress of reactions was monitored by thin-layer chromatography using silica gel 60 F₂₅₄ coated plates (E. Merck). Flash chromatography was performed using ZEOprep™ silica gel 60 (40-63 m) from Canadian Life Science or FlasuPure™ system from Buchi. Detection was carried out under UV light or by spraying with 20% ethanolic sulfuric acid or molybdate or KMnO₄ solution followed by heating. NMR spectra were recorded on Bruker ULTRASHIELD™ 300 MHz and Bruker Avance™III HD 400 and 600 MHz spectrometers. Proton and carbon chemical shifts (6) are reported in ppm relative to the chemical shift of residual CHCl₃, which was set at 7.27 ppm (¹H) and 77.00 ppm (¹³C). Coupling constants (J) are reported in Hertz (Hz), and the following abbreviations are used for peak multiplicities: singlet (s), doublet (d), doublet of doublets (dd), doublet of doublet with equal coupling constants (tₐₚ), triplet (t), multiplet (m). Analysis and assignments were made using COSY (Correlated SpectroscopY) and HSQC (Heteronuclear Single Quantum Coherence) experiments. High-resolution mass spectra (HRMS) were measured with a LC-MS-TOF (Liquid Chromatography Mass Spectrometry Time Of Flight) instrument from Thermo Scientific in positive and/or negative electrospray mode. Either protonated ions (M+H)⁺ or sodium adducts (M+Na)⁺ were used for empirical formula confirmation. LC method: Samples were injected (2 μL) onto an PrePure™ C18 150×4.6 mm column with 5 μm particles (BUCHI) using a Dionex Ultimate™ 3000 system (Thermo Scientific) with water (A) and acetonitrile (B), both containing 0.1% acetic acid, at a flow rate of 800 μL/min at room temperature. The gradient started at 5% B, held for 0.5 min. It was increased to 15% B in 1 minutes, then to 27% B in 14.5 minutes, and then to 95% B in 4 minutes. The gradient was held at 95% B for 2 minutes, and it was then decreased at 5% B in 1 minute. Finally, the gradient was held at 5% B for 1 minute. The total time is 24 minutes. MS method: MS spectra were collected on an TSQ Quantum Access Max™ (Thermo Scientific) equipped with a HESI ion source in positive ion mode set at 4.5 kV source voltage, 320° C. source temperature. MS acquisition was from m z 200-1000 in SCAN mode. The data was analyzed using Thermo XCalibur Qual Browser™.

### Example 1: Synthesis of Allyl 2-acetamido-2-deoxy-α-D-galactopyranoside (2)

Acetyl chloride (4.85 mL, 68.0 mmol, 3.4 equiv.) was added to allylic alcohol (80 mL) under N₂ at 0° C. The solution was stirred at this temperature for one hour and then N-acetylgalactosamine (GalNAc, (1), 4.42 g, 20.0 mmol, 1.0 equiv.) was added to the solution at room temperature. The mixture was stirred at 70° C. for one hour. The solution was cooled to room temperature and then kept at 0° C. The solution was diluted with MeOH (40 mL) and neutralized with solid NaHCO₃ (8.0 g) until reaching pH 7.0. The mixture was filtered under a pad of Celite™ and washed with MeOH. The solvent was then removed under reduced pressure. The dry crude product was either 1) dissolved in water (50 mL) and washed with DCM (4×200 mL) followed with EtOAc (200 mL), the aqueous layer separated and then kept at −80° C. followed the lyophilisation; or 2) precipitated in MeOH/hexane or EtOH/diisopropyl ether; or 3) purified by flash chromatography on silica gel using gradient (EtOAc 100% to EtOAc/MeOH 4:1) to afford desire product allyl GalNAc (2) as white solid (4.18 mg, 1.60 mmol, 80%). Rf=0.30; ACN/H₂O 95:5; ¹H NMR (CD₃OD, 600 MHz): δ 5.99-5.88 (m, 1H, OCH₂CH═CH₂), 5.31 (dd, 1H, Jₜᵣₐₙₛ=17.3, J_{gem}=1.3 Hz, OCH₂CH═CH₂), 5.17 (dd, 1H, J_{cis}=10.5 Hz, OCH₂CH═CH₂), 4.86 (d, 1H, J_{1,2}=3.8 Hz, H-1), 4.27 (dd, 1H, J_{2,3}=11.0 Hz, H-2), 4.20 (m, 1H, OCH₂), 4.00 (m, 1H, OCH₂), 3.89 (dd, J_{3,4}=J_{4,5}=2.6 Hz, H-4), 3.85-3.77 (m, 2H, H-3 and H-5), 3.72 (m, 2H, H-6a and H-6b) and 1.99 ppm (s, 3H, CH₃); ¹³C NMR (CD₃OD, 150 MHz): δ 172.5 (NHCO), 134.2 (OCH₂CH═CH₂), 116.1 (OCH₂CH═CH₂), 96.6 (C-1), 71.2 (C-3), 69.0 (C-4), 68.3 (C-5). 67.8 (OCH₂), 61.4 (C-6), 50.2 (C-2) and 21.2 ppm (CH₃). ESI⁺-HRMS: [M+H]⁺ calcd for C₁₁H₂₀O₆N, 262.1285; found, 262.1294. LC-MS: rt=4.94 min.

### Example 2: Synthesis of Allyl 2-acetamido-4,6-O-benzylidene-2-deoxy-α-D-galactopyranoside (3)

To a solution of allyl GalNAc (2) (2.35 g, 9.0 mmol, 1.0 equiv.) and benzaldehyde dimethylacetal (6.75 mL, 45.0 mmol, 5.0 equiv.) in dry DMF (20 mL) was added a catalytic amount of p-toluenesulfonic acid monohydrate. The mixture was stirred at room temperature. After 5 hours, the mixture was diluted with CHCl₃ and washed with a saturated aqueous solution of NaHCO₃. The organic layer was separated and washed with water, dried over Na₂SO₄, and concentrated to afford a white solid. The benzylidene acetal (compound (3)) was isolated by precipitation in EtOAc/Hexanes as white solid (2.64 g, 7.56, 84%). Rf=0.21; DCM/MeOH 9.0:0.5; ¹H NMR (CDCl₃, 300 MHz): δ 7.59-7.46 (m, 2H, H-ar), 7.43-7.31 (m, 3H, H-ar), 5.91 (m, 1H, OCH₂CH═CH₂), 5.75 (d, 1H, J_{NH,H2}=9.0 Hz, NH), 5.58 (s, 1H, PhCH), 5.34-5.17 (m, 2H, OCH₂CH═CH₂), 5.01 (d, 1H, J₁,2=3.5 Hz, H-1), 4.56-4.42 (ddd, 1H, J_{2,3}=10.9 Hz, J_{2,OH}=9.1 Hz, H-2), 4.34 (dd, 1H, J_{5,6a}=1.5 Hz, J_{6a,6b}=12.5 Hz, H-6a), 4.19 (m, 2H, H-4 and OCH₂), 4.04 (m, 1H, dd, 1H, J_{5,6b}=1.6 Hz, J_{6a,6b}=12.5 Hz, H-6b), 4.01 (m, OCH₂), 3.86 (dd, 1H, J₃,4=10.9 Hz, H-3), 3.71 (sb, 1H, H-5), 2.80 (d, 1H, J_{3,OH}=10.7 Hz, OH-3) and 2.05 ppm (s, 3H, CH₃). ESI⁺-HRMS: [M+H]⁺ calcd for C₁₈H₂₄O₆N, 350.1598; found, 350.1608. LC-MS: rt=19.78 min.

### Example 3: Synthesis of Allyl (2,3,4,6-tetra-O-acetyl-β-D-galactopyranosyl)-(1-→3)-2-acetamido-4,6-O-benzylidene-2-deoxy-α-D-galactopyranoside (5a)

Compound (3) (657 mg, 1.88 mmol, 1.0 equiv.) and 2,3,4,6-tetra-O-benzoyl-α-D-galactopyranosyl bromide (Compound (4b), 3.48 g, 8.46 mmol, 4.5 equiv.) were stirred with silver-exchange zeolite (2.56 g) in anhydrous toluene (60 mL) containing 4 Å molecular sieves (800 mg) under argon atmosphere at 70° C. for 18 hours. The mixture was then filtered off under a pad of Celite™ The solvent was removed under reduced pressure. The crude product was purified by chromatography on silica gel using a gradient of 100% hexanes to hexanes/EtOAc 1:4 to afford the desired disaccharide compound (5a) as white solid (817 mg, 1.20 mmol, 64%). Rf=0.24; hexanes/EtOAc 1:4; ¹H NMR (CDCl₃, 400 MHz): δ 7.57-7.50 (m, 2H, H-ar), 7.42-7.30 (m, 3H, H-ar), 5.97-5.80 (m, 1H, OCH₂CH═CH₂), 5.63-5.545 (m, 3H), 5.38 (dd, 1H, J_{3,4′}=3.3 Hz, J_{4,5′}=1.0 Hz, H-4^{II}), 5.33-5.14 (m, 4H), 5.05 (d, 1H, J=3.5 Hz, H-1), 5.11-5.00 (m, 1H, H-2), 4.76 (d, 1H, J=7.9 Hz, H-1′), 4.68 (m, 1H), 4.30-3.80 (m, 7H), 3.65 (m, 1H), 2.15 (s, 3H), 2.04 (s, 6H), 1.99 (s, 3H) and 1.97 ppm (s, 3H). ESI⁺-HRMS: [M+H]⁺ calcd for C₃₂H₄₂O₁₅N, 680.2549; found, 680.2549 (0.0 ppm). LC-MS: rt=13.11 min.

### Example 4: Synthesis of Allyl (2,3,4,6-tetra-O-benzoyl-β-D-galactopyranosyl)-(1→3)-2-acetamido-4,6-O-benzylidene-2-deoxy-α-D-galactopyranoside (5b)

Compound (3) (101 mg, 0.29 mmol, 1.0 equiv.) and 2,3,4,6-tetra-O-benzoyl-α-D-galactopyranosyl bromide (Compound (4b), 239 mg, 0.58 mmol, 2.0 equiv.) were stirred with silver-exchange zeolite (430 mg) in anhydrous toluene (10 mL) containing 4 Å molecular sieves under argon atmosphere at 70° C. for 18 hours. The mixture was then filtered off under a pad of Celite™. The solvent was removed under reduced pressure. The crude product was purified by chromatography on silica gel using a gradient of 100% hexanes to hexanes/EtOAc 1:2 to afford the desired disaccharide compound (5b) as white solid (250 mg, 0.27 mmol, 94%). mp: 110-111° C.; Rf=0.20; hexanes/EtOAc 1:2; ¹H NMR (CDCl₃, 600 MHz): δ 8.06-7.19 (m, 25H, H-ar), 5.98 (dd, 1H, J₃,4, =3.3 Hz, J₄,5, =1.0 Hz, H-4^{II}), 5.85-5.78 (m, 2H, OCH₂CH═CH₂ and H-2^{II}), 5.60 (dd, 1H, J₂,3, =10.2 Hz, J₃,4, =3.4 Hz, H-3^{II}), 5.48 (sb, 1H, NH), 5.23 (m, 3H, OCH₂CH═CH₂ and H-1), 4.68 (dd, 1H, J_{5′,6a′}=6.9 Hz, J_{6a′,6b′}=11.4 Hz, H-6a^{I}), 4.63-4.58 (m, 1H, H-2), 4.46-4.36 (m, 3H, H-4. H-5 and H-6b^{II}), 4.14-4.07 (m, 3H, H-6a, OCH₂ and H-3), 3.96 (m, 1H, OCH₂), 3.75 (m, 1H, H-6b), 3.51 (m, 1H, H-5) and 1.40 ppm (s, 3H, CH₃); ¹³C NMR (CDCl₃, 150 MHz): δ 170.0 (NHCO), 166.0, 165.5, 165.4, 165.2 (CO), 137.6-126.2 (multi, 30 C-arom), 133.2 (OCH₂CH═CH₂), 117.8 (OCH₂CH═CH₂), 102.0 (C-1^{II}), 100.9 (CPhCH), 97.3 (C-1^{I}), 76.1 (C-3), 75.4 (C-4), 71.7 (C-3^{II} and C-5^{II}), 70.2 (C-2^{II}), 69.1 (C-6), 68.6 (OCH₂), 68.1 (C-4^{II}), 62.9 (C-5), 62.6 (C-6^{I}), 48.2 (C-2) and 22.5 ppm (CH₃). ESI⁺-HRMS: [M+H]⁺ calcd for C₅₂H₅₀O₁₅N, 928.3175; found, 928.3133.

### Example 5: Synthesis of Allyl (β-D-galactopyranosyl)-(1→3)-2-acetamido-2-deoxy-α-D-galactopyranoside (6)

A solution of compound (5b) (1.12 g, 1.20 mmol, 1.0 equiv.) in 1M sodium methoxide in methanol (12 mL, pH 8-9) was stirred at room temperature until consumption of starting material. After 1.5 hours, the solution was neutralized by the addition of ion-exchange resin (Amberlite™ IR 120, H⁺), filtered, washed with MeOH, and the solvent removed under reduced pressure to afford the intermediate as white solid. The white solid intermediate was then dissolved in 10 mL of 60% aqueous acetic acid and the resulting solution was stirred at 60° C. for 1.5 hours. The solvent was removed under reduced pressure, and the residue was dissolved in water and washed several times with dichloromethane and twice with EtOAc. The water layer was then lyophilized to afford the deprotected allyl compound (6) as white solid (400 mg, 0.94 mmol, 79%). mp=230-232° C.; Rf=0.53; CHCl₃/MeOH/H₂O 11:6:1; ¹H NMR (D₂O, 600 MHz): δ 5.80 (m, 1H, OCH₂CH═CH₂), 5.19 (dd, 1H, Jₜᵣₐₙₛ=17.3 Hz, OCH₂CH═CH₂), 5.09 (dd, 1H, J_{cis}=10.4 Hz, OCH₂CH═CH₂), 4.77 (d, 1H, J_{1,2}=3.7 Hz, H-1), 4.29 (d, 1H, J_{1,2}=3.7 Hz, H-1), 4.29 (d, 1H, J_{1,2}=7.8 Hz, H-1^{II}), 4.16 (dd, 1H, J_{2,3}=11.2 Hz, J_{1,2}=3.7 Hz, H-2), 4.08-4.01 (m, 2H, H-4 and OCH₂), 3.92-3.82 (m, 3H, H-3, H-5 and OCH₂), 3.73 (dd, 1H, H-4^{II}), 3.63-3.52 (m, 4H, H-6a,b and H-6′a,b), 3.47 (m, 2H, H-3^{II} and H-5^{II}), 3.39 (dd, 1H, J_{2′,3′}=10.0 Hz, J_{1′,2}′=7.7 Hz, H-2^{II}) and 1.85 ppm (s, 3H, CH₃); ¹³C NMR (D₂O, 150 MHz): δ 174.6 (NHCO), 133.7 (OCH₂CH═CH₂), 117.9 (OCH₂CH═CH₂), 104.7 (C-1^{II}, 96.4 (C-1), 77.2 (C-3), 75.0 (C-5^{II}, 72.5 (C-3^{II}, 70.7 (C-5), 70.6 (C-2^{II}, 68.8 (C-4), 68.6 (C-4^{II}, 68.4 (OCH₂), 61.2 (C-6^{II}, 61.0 (C-6), 48.6 (C-2) and 22.0 ppm (CH₃). ESI⁺-HRMS: [M+Na]⁺ calcd for C₁₇H₂₉O₁₁NNa, 446.1633; found, 446.1613. LC-MS: rt=4.77 min.

### Example 6: N-Succinimidyl-3-{[3-(β-D-galactopyranosyl)-(1-*3)-2-acetamido-2-deoxy-α-D-galactopyranosyl)oxypropyl]thio}propanoate (11b) and Conjugation with Protein CRM197 to form Glycoconjugate (II)

Compound (6) (138 mg, 0.53 mmol, 1.0 equiv.), compound (7) (538 mg, 2.65 mmol, 5 equiv. —commercially available from Sigma-Aldrich) and 4,4′-azobis(4-cyanovaleric acid) (ACVA, 1.5 mg, 0.05 mmol, 0.1 equiv.) were stirred in degassed acetonitrile (5 mL) at reflux (70° C.) for 2 hours. After returning to room temperature, the solvent was removed under reduced pressure. The Compound (6) and compound (7) can also reactivate with DMPA (0.1 eq) in THF/water (1:1; v/v) under UV irradiation (280-320 nm) at 25° C. for 20 minutes. The crude product was suspended in water and extraction by washing several times with dichloromethane and twice EtOAc to remove succinimide reagent. The final aqueous layer containing crude compound (11b; NHS-TF) was cooled down to −80° C. to lyophilise. Then, the crude compound (11b), without any further purification, was used directly in the conjugation step with the protein CRM197 to afford glyconjugate (II; CRM-TF). Also, the crude white solid was purified by reverse preparative HPLC on C-18 to afford the white solid 11b. The pure compound (11b) was used in the conjugation step with the protein CRM197 to afford glyconjugate (II; CRM-TF). ¹H NMR (D₂O, 400 MHz): δ 4.83 (d, 1H, J=3.7 Hz, H-1), 4.40 (d, 1H, J=7.7 Hz, H-1^{II}), 4.25 (dd, 1H, J=17.0, 6.9 Hz), 4.18 (d, 1H, J=2.6 Hz), 4.02-3.90 (m, 2H), 3.84 (d, 1H, J=3.1 Hz), 3.80-3.62 (m, 5H), 3.62-3.40 (m, 4H), 3.02 (t, 2H, J=6.7 Hz), 2.96-2.83 (m, 5H), 2.79-2.55 (m, 3H), 1.96 (s, 3H) and 1.87 (dd, 2H, J=12.9, 6.4 Hz); ¹³C NMR (D₂O, 100 MHz,): δ 176.6, 174.6, 173.3, 169.2, 104.8, 97.2, 77.3, 75.0, 72.6, 70.6, 68.8, 68.6, 66.4, 66.3, 61.2, 61.0, 48.7, 34.3, 31.4, 28.4, 28.2, 26.3, 25.7, 25.6, 25.2, and 22.0 ppm (CH₃). ESI⁺-HRMS: [M+Na]⁺ calcd for C₂₄H₃₈O₁₅N₂SNa, 649.18851; found, 649.18838, 0.2 ppm. HPLC: Tr=10.747.

For the conjugation step, the carrier protein was buffer exchanged to PBS at pH 8 by centrifugal filtration (Amicon™ MWCO; 10 or 30K) and its concentration adjusted to 2 mg/ml. A Bradford assay with BSA as a standard was used to measure the protein concentration. The conjugation to the protein was initiated by adding a fresh 20 mM solution of compound (11b) in water to the protein to reach a final protein concentration of 1 mg/ml. The amount of compound (IIb) to be added to the protein can be adjusted depending on the protein's number of surface accessible lysines and the final ratio of conjugation that is intended. Then, the solution was vortexed for 90 minutes then washed by centrifugal filtration using PBS at pH 7.4 (Amicon™ MWCO; 10 or 30K), or gel filtration.

The conjugation of the protein to compound (11b), i.e. the obtention of glyconjugate (II), was demonstrated by ELISA, Western blot, MALDI-TOF and Bradford assay.

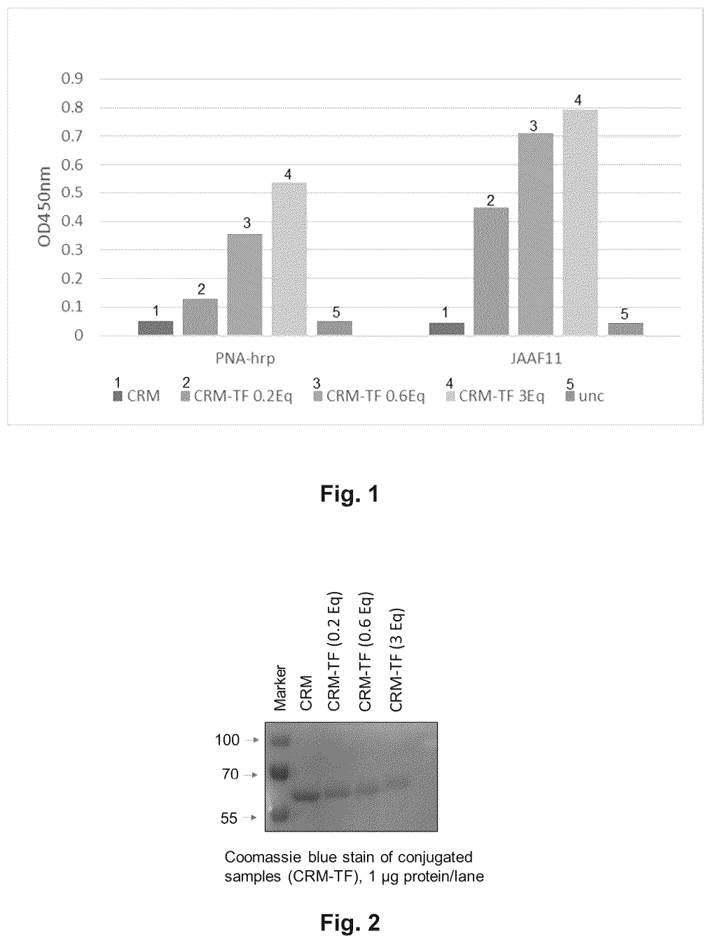

The reactivity of the TF-specific lectin Peanut Agglutinin (PNA) and anti-TF monoclonal antibody JAAF11 to 0.01 μg of unconjugated CRM197 and CRM197 conjugated to TF (CRM-TF: glyconjugate (II)) at 3 different ratios (0.2, 0.6, 3) of NHS-TF (compound (11b)) to the protein's total number of lysine was determined by ELISA. As shown in FIG. 1, the ELISA indicates a correlation between the conjugation conditions and the reactivity of the resulting glycoconjugate.

FIG. 2 shows the Coomassie stained SDS-PAGE gel of 1 μg of unconjugated CRM197 and CRM197 conjugated to TF (CRM-TF: glyconjugate (II)) at 3 different ratios of NHS-TF (compound (11b)) to the protein's total number of lysines. The observed decrease in mobility of the glycoconjugate bands in function of the conjugation's conditions relative to the unconjugated CRM indicate that the CRM is conjugated.

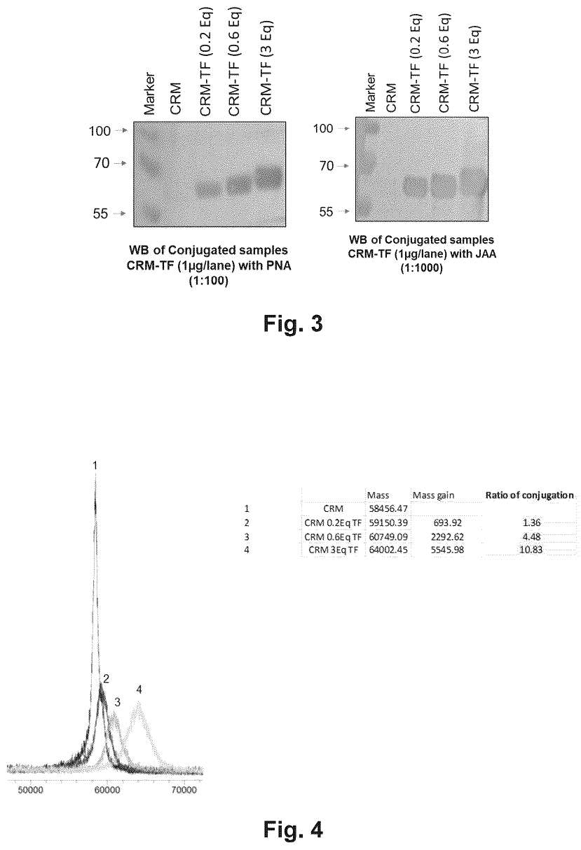

A Western blot of 1 μg of unconjugated CRM197 and CRM197 conjugated to TF (CRM-TF: glyconjugate (II)) at 3 different ratios of NHS-TF (compound (11b)) to the protein's total number of lysines detected was recorded using the TF-specific lectin Peanut Agglutinin (PNA) and anti-TF monoclonal antibody JAAF11. As shown in FIG. 3, the Western blot reveals reactive bands to both PNA and JAAF11 of increasing molecular weight in relation to the conjugation conditions indicating that CRM is conjugated and an absence of reactivity to unconjugated CRM.

MALDI-TOF spectra of unconjugated CRM197 and CRM197 conjugated to TF (CRM-TF: glyconjugate (II)) at 3 different ratios of NHS-TF (compound (11b)) to the protein's total number of lysines were recorded. As shown in FIG. 4, the spectra indicate an increase of mass in function of the conditions of conjugation. The table in inset shows the gain of mass and the average molar ratio of conjugation to TF.

### Example 7: N-Succinimidyl-3-[3-(2-acetamido-2-deoxy-α-D-galactopyranosyloxypropyl)thio]-propanoate (11a) and Conjugation with Protein CRM197 to Form Glycoconjugate (I)

Compound (2) (200 mg, 0.765 mmol, 1.0 equiv.), compound (7) (933 mg, 4.59 mmol, 6.0 equiv.) and 4,4′-azobis(4-cyanovaleric acid) (ACVA, 2 mg, 0.08 mmol, 0.01 equiv.) were stirred in degassed acetonitrile and isopropanol (4.0 mL, 1:1) at reflux (70° C.) for 2 hours. After returning to room temperature, the solvent was removed under reduced pressure. The crude product was suspended in water and purified by A) chromatography on C18 silica gel or C18 HPLC or B) extraction by washing several times with dichloromethane and twice EtOAc to remove succinimide reagent. The final aqueous layer containing crude compound (11a; NHS-TN) was cooled down to −80° C. to lyophilise. Then, the crude compound (11a), without any further purification, was used directly in the conjugation step with the protein CRM197 to afford glyconjugate (I; CRM-TN).

For the conjugation step, the carrier protein was buffer exchanged to PBS at pH 8 by centrifugal filtration (Amicon™ MWCO; 10 or 30K) and its concentration was adjusted to 2 mg/ml. A Bradford assay with BSA as a standard was used to measure the protein concentration. The conjugation to the protein was initiated by adding a fresh 20 mM solution of compound (11a) in water to the protein to reach a final protein concentration of 1 mg/ml. The amount of compound (11a) to be added to the protein can be adjusted depending on the protein's number of surface accessible lysines and the final ratio of conjugation that is intended. Then, the solution was vortexed for 90 minutes then washed by centrifugal filtration using PBS at pH 7.4 (Amicon™ MWCO; 10 or 30K), or gel filtration.

1. A process for synthesizing a reactive carbohydrate of Formula (A) 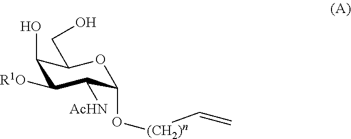
where R¹ is H and n is an integer from 1 to 5,
comprising reacting, under heating, a compound of Formula (A₀) 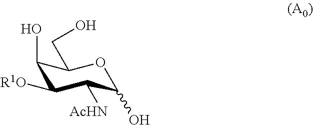
with HO—(CH₂)ₙ—CH═CH₂ in the presence of an acid capable of liberating a proton, resulting in a reaction mixture comprising the compound of Formula (A), and
cooling the reaction mixture to obtain a cooled mixture comprising the compound of Formula (A).

2. The process of claim 1, wherein the acid comprises acetyl chloride, acetic acid, an acidic cation exchange resin, camphorsulfonic acid, p-toluenesulfonic acid monohydrate, HCl or any mixture thereof.

3. The process of claim 1 or 2, wherein the acid comprises acetyl chloride.

4. The process of any one of claims 1 to 3, wherein reacting the compound of Formula (A₀) comprises adding the compound of Formula (A₀) to a solution comprising HO—(CH₂)ₙ—CH═CH₂ and acetyl chloride at a mixing temperature from about 0° C. to about 25° C. resulting in an intermediate reaction mixture at low temperature comprising the compound of Formula (A₀), HO—(CH₂)ₙ—CH═CH₂ and HCl.

5. The process of claim 4, wherein the mixing temperature is about 0° C., or about 0° C. to about 10° C., or about 0° C. to about 20° C., or about 10° C. to about 20° C., about 10° C. to about 25° C.

6. The process of claim 4 or 5, wherein the solution comprising HO—(CH₂)ₙ—CH═CH₂ and acetyl chloride is prepared at a temperature of about 0° C. under inert atmosphere.

7. The process of any one of claims 4 to 6, wherein reacting the compound of Formula (A₀) comprises contacting the intermediate reaction mixture with a heat source at a temperature of from about 40° C. to about 80° C.

8. The process of any one of claims 1 to 7, wherein the heating is performed at a reaction temperature from about 40° C. to about 80° C. for a reaction time of from about 30 minutes to 5 hours.

9. The process of claim 8, wherein the reaction temperature is from about 60° C. to about 80° C.

10. The process of claim 8 or 9, wherein the reaction temperature is about 70° C.

11. The process of any one of claims 8 to 10, wherein the reaction time is from about 30 min to about 1 hour.

12. The process of any one of claims 8 to 11, wherein the reaction time is from about 30 min to about 45 min.

13. The process of any one of claims 8 to 12, wherein the reaction time is about 30 min.

14. The process of claim 1 or 2, wherein the acid comprises acetic acid, an acidic cation exchange resin, camphorsulfonic acid, p-toluenesulfonic acid monohydrate, HCl or any mixture thereof.

15. The process of claim 14, wherein reacting the compound of Formula (A₀) with HO—(CH₂)ₙ—CH═CH₂ comprises heating a solution comprising the compound of Formula (A₀), HO—(CH₂)ₙ—CH═CH₂ and the acid.

16. The process of claim 15, wherein the solution further comprises a solvent selected from the group consisting of dichoromethane, chloroform, 1,3-dioxolane, diethoxymethane, dimethoxymethane, 2,5,7,10-tetraoxaundecane, dipropoxymethane and any combination thereof.

17. The process of claim 15 or 16, wherein the reaction is performed at a temperature of from about 30° C. to about 70° C.

18. The process of any one of claims 15 to 17, wherein the reaction is performed for a reaction time of about 0.5 hour to about 15 hours, or from about 0.5 hour to about 10 hours, or from about 0.5 hour to about 5 hours, or from about 0.5 hour to about 2 hours.

19. The process of any one of claims 1 to 18, further comprising neutralizing the cooled mixture.

20. The process of any one of claims 1 to 19, further comprising isolating the compound of Formula (A).

21. A process for synthesizing a reactive carbohydrate of Formula (AA_{PG}) 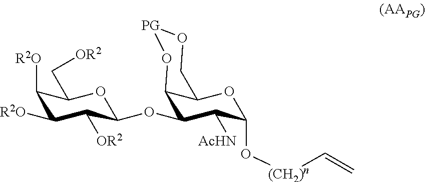
where R² is a monovalent protecting group, PG is a divalent protecting group and n is an integer from 1 to 5,
comprising reacting, in the presence of a metal-containing zeolite, a compound of Formula (A_{PG}) 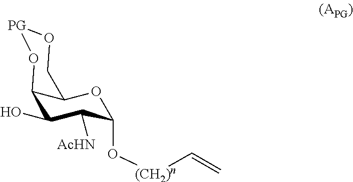
with a compound of Formula (A₁) 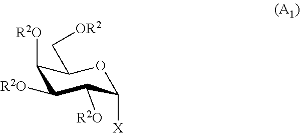
where X is —I, —Br, —Cl, —CN, —OTf (Triflate), —OMs (Mesylate), —OTs (Tosylate), methylsulfate, or trichloroacetimidate.

22. The process of claim 21, wherein the metal in the metal-containing zeolite comprises Ag, Al, Cd, Co, Cu, Fe, Ga, In, Mo, Pd, Pt, Sn, Sb, V, Zr or any mixture thereof.

23. The process of claim 21, wherein the metal-containing zeolite comprises a zeolite selected from the group consisting of Sn-Beta, Zr-Beta, Al-Beta(OH), Al-Beta(F), Pt@MCM-22, K-PtSn/MFI, 0.3Pt/0.5Sn—Si-Beta, Pt/Sn 2.0-Beta, 0.5CoSi-Beta, V-Beta, H—[Fe]ZSM-5, Fe-BEA, Ga-Beta, Ga-Beta-200, Mo/HZSM-5, or any mixture thereof.

24. The process of claim 21, wherein the metal-containing zeolite comprises a silver-containing zeolite.

25. The process of any one of claims 21 to 24, wherein the reaction is performed in an organic solvent selected from the group consisting of toluene, dichorometane-toluene mixture, DMSO-toluene mixture, and N-methylpyrrolidone-toluene mixture.

26. The process of any one of claims 21 to 25, wherein the reaction is performed in toluene.

27. The process of any one of claims 21 to 26, wherein the reaction is performed in the presence of a molecular sieve.

28. The process of any one of claims 21 to 27, wherein the reaction is performed in the presence of a molecular sieve of type 3 Å, type 4 Å, type 5 Å, type 13X or any mixture thereof.

29. The process of any one of claims 21 to 28, wherein the reaction is performed at a temperature of from about 20° C. to about 80° C.

30. The process of any one of claims 21 to 29, wherein the reaction is performed at a temperature of from about 20° C. to about 50° C.

31. The process of any one of claims 21 to 30, wherein the reaction is performed at a temperature of from about 20° C. to about 30° C.

32. The process of any one of claims 21 to 31, wherein the reaction is performed for about 5 hours to about 50 hours.

33. The process of any one of claims 21 to 32, wherein the reaction is performed for about 5 hours to about 30 hours.

34. The process of any one of claims 21 to 31, wherein the reaction is performed at about 60 to about 80° C. for about 15 hours to about 20 hours.

35. The process of any one of claims 21 to 31, wherein the reaction is performed at about 20° C. to about 25° C. for about 20 hours to about 30 hours.

36. The process of any one of claims 21 to 35, wherein R² is selected from the group consisting of Ac, Bz, allyl, benzoate, methoxymethyl (MOM), tetrahydropropyranyl (THP), t-butyl, pivalate, t-butyldimethylsilyl (TBDMS) and t-butyldiphenylsilyl (TBDPS).

37. The process of any one of claims 21 to 36, wherein R² is Ac or Bz.

38. The process of any one of claims 21 to 37, wherein PG is selected from the group consisting of PhCH, Me₂C, O═C, Me₂Si and 2,6-bis(trifluoromethyl)phenyl-B.

39. The process of any one of claims 21 to 38, wherein PG is PhCH.

40. The process of any one of claims 21 to 39, wherein X is Br.

41. The process of any one of claims 21 to 40, wherein the reaction is performed under inert atmosphere.

42. The process of any one of claims 21 to 41, further comprising isolating the compound of Formula (AA_{PG}).

43. The process of any one of claims 21 to 42, wherein the compound of Formula (A_{PG}) is formed by protecting a compound of Formula (A) 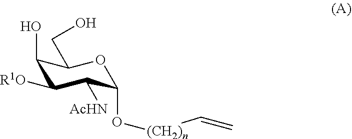
where R¹ is H, with the protecting group PG.

44. The process of claim 43, wherein protecting the compound of Formula (A) is performed by reaction with a compound selected from the group consisting of PhCH(OMe)₂, triphosgene, Me₂SiCl₂, Me₂C(OMe)₂, 2-methoxypropene and 2,6-bis(trifluoromethyl)phenylboronic ester.

45. The process of claim 43 or 44, wherein protecting the compound of Formula (A) is performed in an organic solvent selected from the group consisting of DMF, Dimethyl acetamide (DMAC), DMSO, THF, Dioxane, 1-3 Dioxolane, acetonitrile, 2,5-dimethyl tetrahydrofuran (DMTHF), Gamma-vaterolactone (GVL), Dihydrolevoglucoserone (Cyrene), methyl levulinate (ML), Ethyl levulinate (EL), Ethyl levulinate propyleneglycol ketal (ELPK), Dimethyl glutarate (DMG), Dimethylpropylene urea (DMPU), Poly(propyleneglycol) (PPG), Glycofurol (THFP), 1-Ethyl-3-methylimidazolium acetate ([emim][OAc]) and any combination thereof.

46. The process of any one of claims 43 to 45, wherein the compound of Formula (A) is obtained by the process of any one of claims 1 to 20.

47. A process for synthesizing a reactive carbohydrate of Formula (AA) 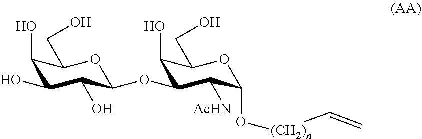
where n is an integer from 1 to 5,
comprising deprotecting the compound of Formula (AA_{PG}) obtained by the process of any one of claims 21 to 46.

48. The process of claim 47, wherein deprotecting comprises reacting the compound of Formula (AA_{PG}) with a basic solution to form an intermediate product, and then treating the intermediate product with an acidic solution.

49. The process of claim 48, wherein the basic solution comprises a base is selected from the group consisting of a methoxide, ethoxide and 2-methylpropan-2-olate, preferably the base is sodium methoxide.

50. The process of claim 48 or 49, wherein the basic solution comprises a solvent selected from the group consisting of methanol, ethanol and isopropanol, preferably the solvent is methanol.

51. The process of any one of claims 48 to 50, wherein the acidic solution comprises an acetic acid-water mixture or an ethanolic acid-water mixture, preferably the acidic solution is an aqueous acetic acid solution.

52. The process of any one of claims 48 to 51, wherein the reaction with the basic solution is performed at a temperature of from about 0° C. to about 50° C., preferably at room temperature.

53. The process any one of claims 48 to 52, wherein the reaction with the basic solution is performed for a period of about 0.5 hour to about 5 hours, preferably about 1 hour to about 2 hours.

54. The process of any one of claims 48 to 53, wherein treating with the acidic solution is performed at a temperature of from about 50° C. to about 70° C., preferably at about 60° C.

55. The process of any one of claims 48 to 54, wherein treating with the acidic solution is performed for a period of about 1 hour to about 5 hours, preferably about 1 hour to about 2 hours.

56. The process of any one of claims 48 to 55, further comprising neutralizing the basic solution after reacting with the compound of Formula (AA_{PG}) and evaporating any liquid to afford the intermediate product in crude form.

57. The process of any one of claims 48 to 56, wherein the intermediate product is crude when treated with the acidic solution.

58. A process for synthesizing a reactive carbohydrate of Formula (AA) 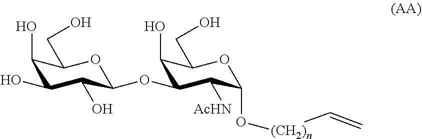
comprising a deprotection a compound of Formula (AA_{PG}) 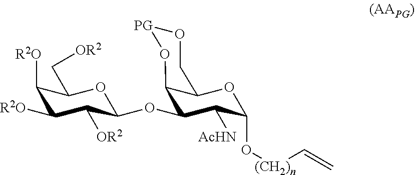
where n is an integer from 1 to 5, R² is a monovalent protecting group, and PG is a divalent protecting group,
the deprotection comprising reacting the compound of Formula (AA_{PG}) with a basic solution to form an intermediate crude product, and then treating the intermediate crude product with an acidic solution.

59. The process of claim 58, wherein the basic solution comprises a base selected from the group consisting of a methoxide, ethoxide and 2-methylpropan-2-olate, preferably the base is sodium methoxide.

60. The process of claim 58 or 59, wherein the basic solution comprises a solvent selected from the group consisting of methanol, ethanol and isopropanol, preferably the solvent is methanol.

61. The process of any one of claims 58 to 60, wherein the acidic solution comprises an acetic acid-water mixture or an ethanolic acid-water mixture, preferably the acidic solution is an aqueous acetic acid solution.

62. The process any one of claims 58 to 61, wherein the reaction with the basic solution is performed at a temperature of from about 0° C. to about 50° C., preferably at room temperature.

63. The process any one of claims 58 to 62, wherein the reaction with the basic solution is performed for a period of about 0.5 hour to about 5 hours, preferably about 1 hour to about 2 hours.

64. The process of any one of claims 58 to 63, wherein treating with the acidic solution is performed at a temperature of from about 50° C. to about 70° C., preferably at about 60° C.

65. The process of any one of claims 58 to 64, wherein treating with the acidic solution is performed for a period of about 1 hour to about 5 hours, preferably about 1 hour to about 2 hours.

66. The process of any one of claims 58 to 65, further comprising neutralizing the basic solution after reacting with the compound of Formula (AA_{PG}) and evaporating any liquid to afford the intermediate crude product.

67. The process of any one of claims 58 to 66, wherein R² is Ac or Bz, preferably Bz.

68. The process of any one of claims 58 to 67, wherein PG is selected from the group consisting of PhCH, Me₂C, O═C, Me₂Si and 2,6-bis(trifluoromethyl)phenyl-B.

69. The process of any one of claims 58 to 68, wherein PG is PhCH.

70. A process for synthesizing a reactive carbohydrate of Formula (B) or Formula (BB) 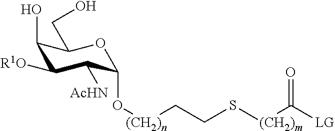
(B) R¹ H
(BB): R¹ is 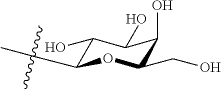
where n is an integer from 1 to 5, m is an integer from 1 to 5 and LG is a leaving group enabling an amido bond formation when the compound of Formula (B) or (BB) is reacted with an amino group,
comprising reacting a compound of Formula (C) 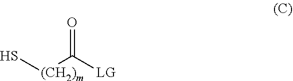
with a compound of Formula (A) 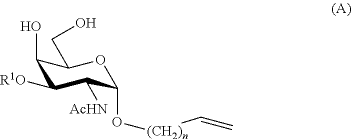
where R¹ is H, to form the compound of Formula (B)
or
with a compound of Formula (AA) 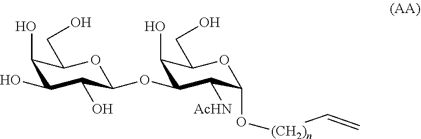
to form the compound of Formula (BB).

71. The process of claim 70, wherein reacting the compound of Formula (C) with the compound of Formula (A) or (AA) is performed in the presence of a photoinitiator.

72. The process of claim 71, wherein the photoinitiator is selected from the group consisting of a free radical-generating azo compound, lithium phenyl-2,4,6-trimethylbenzoylphosphinate (LAP), metals or metal ions-based photoinitiator, peroxides, ammonium persulfate, 2,2-dimethoxy-2-phenylacetophenone (DMPA), and any combination thereof.

73. The process of claim 72, wherein the free radical-generating azo compound is selected from the group consisting of azobisisobutyronitrile (AIBN); 2,2′-azobis(2-methylpropionitrile); 4,4′-azobis(4-cyanopentanoic acid) (ACVA); 1,1′-azobis(cyanocyclohexane) (ACHN); diazenedicarboxylic acid bis(N,N-dimethylamide) (TMAD); azodicarboxylic acid dipiperidide (ADD); 2,2′-azobis[2-(2-imidazolin-2-yl)propane]dihydrochloride; 2,2′-azobis(2-amidinopropane) dihydrochloride (AAPH); 2,2′-azobis(2-methylpropionitrile); 4,4′-(diazene-1,2-diyl)bis(4-cyanopentanoic acid); 2,2′-azodi(2-methylbutyronitrile); and any combination thereof.

74. The process of claim 72 or 73, wherein the peroxide is selected from the group consisting of tert-butyl peroxyisobutyrate, tert-butyl hydroperoxide, benzoyl peroxide and any combination thereof.

75. The process of any one of claims 70 to 74, wherein reacting the compound of Formula (C) with the compound of Formula (A) or (AA) is performed in water or an organic solvent.

76. The process of any one of claims 70 to 75, wherein reacting the compound of Formula (C) with the compound of Formula (A) or (AA) is performed in a solvent selected from the group consisting of water, dioxane, acetonitrile, tetrahydrofuran (THF), diisopropyl ether, isopropanol, chlorobenzene, methyl-tert-butyl ether, methanol, ethanol, tert-butanol, chloroform and any combination thereof.

77. The process of any one of claims 70 to 76, wherein reacting the compound of Formula (C) with the compound of Formula (A) or (AA) is performed at a temperature ranging from about 40° C. to about 110° C.

78. The process of any one of claims 70 to 77, wherein reacting the compound of Formula (C) with the compound of Formula (A) or (AA) is performed for a period of about 1 hour to about 10 hours.

79. The process of claim 70, wherein reacting the compound of Formula (C) with the compound of Formula (A) or (AA) is performed under visible light in the presence of a visible light absorbing transition metal photocatalyst.

80. The process of claim 70, wherein reacting the compound of Formula (C) with the compound of Formula (A) or (AA) is performed under ultraviolet light irradiation.

81. The process of claim 70, wherein reacting the compound of Formula (C) with the compound of Formula (A) or (AA) is performed under ultraviolet light irradiation in the presence of a photoinitiator.

82. The process of claim 81, wherein the photoinitiator is selected from the group consisting of 2,2′-azidobis[2-imidazolin-2-yl)propane]dihydrochloride; 2,2-dimethoxy-2-phenylacetophenone (DMPA); and combination thereof.

83. The process of any one of claims 80 to 82, wherein reacting the compound of Formula (C) with the compound of Formula (A) or (AA) is performed in an alcoholic solvent such as methanol, ethanol, isopropanol or any combination thereof.

84. The process of any one of claims 80 to 83, wherein reacting the compound of Formula (C) with the compound of Formula (A) or (AA) is performed at a temperature ranging from about 20° C. to about 30° C.

85. The process of any one of claims 80 to 84, wherein reacting the compound of Formula (C) with the compound of Formula (A) or (AA) is performed for a period of about 1 hour to about 24 hours.

86. The process of any one of claims 80 to 85, wherein the ultraviolet light irradiation is short-wave, medium-wave or long-wave ultraviolet light irradiation.

87. The process of any one of claims 70 to 86, wherein m is an integer from 1 to 3.

88. The process of any one of claims 70 to 87, wherein m is 2.

89. The process of any one of claims 70 to 88, wherein LG is an O-fluorophenyl group such as OPhF₅ or OPhF₄(para SO₃Na), or an O—(N-succinimidyl) group.

90. The process of any one of claims 70 to 89, wherein LG is an O—(N-succinimidyl) group.

91. The process of any one of claims 70 to 90, wherein the compound of Formula (A) is obtained by the process of any one of claims 1 to 20.

92. The process of any one of claims 70 to 90, wherein the compound of Formula (AA) is obtained by the process of any one of claims 47 to 69.

93. A process for preparing a glycoconjugate of Formula (I) or (II) 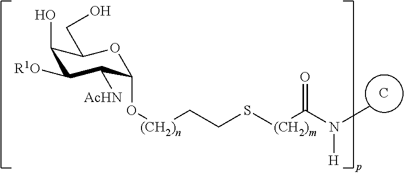
(I): R¹═H
(II): R¹═ 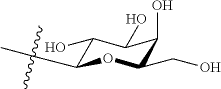
where n is an integer from 1 to 5, m is an integer from 1 to 5, p is an integer from 1 to 50 and 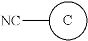
is a carrier material containing at least one amino group available for conjugation, comprising conjugating at least one free amino group of the carrier material with a compound of Formula (B) to form the glycoconjugate of Formula (I) or with a compound of Formula (BB) to form the glycoconjugate of Formula (I), wherein the compounds of Formula (B) and (BB) are obtained by the process of any one of claims 70 to 92.

94. The process of claim 93, wherein the carrier material comprises a protein, polypeptide or peptide.

95. The process of any one of claims 1 to 94, wherein n is an integer from 1 to 3.

96. The process of any one of claims 1 to 95, wherein n is 1 or 2.

97. The process of any one of claims 1 to 96, wherein n is 1.

98. A compound of Formula (AA_{PG}) 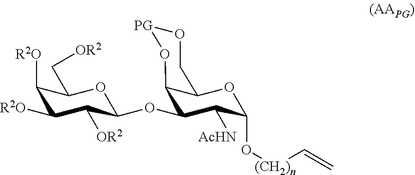
where n is an integer from 1 to 5, R² is Ac and PG is a divalent protecting group selected from PhCH, Me₂C, O═C, Me₂Si and 2,6-bis(trifluoromethyl)phenyl-B.

99. The compound of claim 98, wherein PG is PhCH.

100. The compound of claim 98 or 99, wherein n is an integer from 1 to 3.

101. The compound of any one of claims 98 to 100, wherein n is 1 or 2.

102. The compound of any one of claims 98 to 101, wherein n is 1.

103. The compound of claim 98, having the formula:

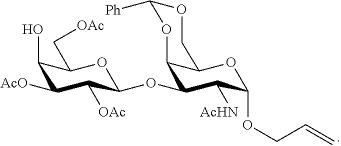

104. A compound of formula: [chemical structure]
